SEQUENCE LISTING (28 sequences)

SEQ ID NO: 1 - DNA, 5316 bp, Homo sapiens
atcatggcggatggccccaggtgtaagcgcagaaagcaggcgaacccgcggcgcaataacgttacaaattataatactgtggtagaaacaaattcagattcagatgatgaagacaaactgcatattgtggaagaagaaagtgttacagatgcagctgactgtgaaggtgtaccagaggatgacctgccaacagaccagacagtgttaccagggaggagcagtgaaagagaagggaatgctaagaactgctgggaggatgacagaaaggaagggcaagaaatcctggggcctgaagctcaggcagatgaagcaggatgtacagtaaaagatgatgaatgcgagtcagatgcagaaaatgagcaaaaccatgatcctaatgttgaagagtttctacaacaacaagacactgctgtcatttttcctgaggcacctgaagaggaccagaggcagggcacaccagaagccagtggtcatgatgaaaatggaacaccagatgcattttcacaattactcacctgtccatattgtgatagaggctataaacgctttacctctctgaaagaacacattaaatatcgtcatgaaaagaatgaagataactttagttgctccctgtgcagttacacctttgcatacagaacccaacttgaacgtcacatgacatcacataaatcaggaagagatcaaagacatgtgacgcagtctgggtgtaatcgtaaattcaaatgcactgagtgtggaaaagctttcaaatacaaacatcacctaaaagagcacttaagaattcacagtggagagaagccatatgaatgcccaaactgcaagaaacgcttttcccattctggctcctatagctcacacataagcagtaagaaatgtatcagcttgatacctgtgaatgggcgaccaagaacaggactcaagacatctcagtgttcttcaccgtctctttcagcatcaccaggcagtcccacacgaccacagatacggcaaaagatagagaataaaccccttcaagaacaactttctgttaaccaaattaaaactgaacctgtggattatgaattcaaacccatagtggttgcttcaggaatcaactgttcaacccctttacaaaatggggttttcactggtggtggcccattacaggcaaccagttctcctcagggcatggtgcaagctgttgttctgccaacagttggtttggtgtctcccataagtatcaatttaagtgatattcagaatgtacttaaagtggcggtagatggtaatgtaataaggcaagtgttggagaataatcaagccaatcttgcatccaaagaacaagaaacaatcaatgcttcacccatacaacaaggtggccattctgttatttcagccatcagtcttcctttggttgatcaagatggaacaaccaaaattatcatcaactacagtcttgagcagcctagccaacttcaagttgttcctcaaaatttaaaaaaagaaaatccagtcgctacaaacagttgtaaaagtgaaaagttaccagaagatcttactgttaagtctgagaaggacaaaagctttgaagggggggtgaatgatagcacttgtcttctgtgtgatgattgtccaggagatattaatgcacttccagaattaaagcactatgacctaaagcagcctactcagcctcctccactccctgcagcagaagctgagaagcctgagtcctctgtttcatcagctactggagatggcaatttgtctcctagtcagccacctttaaagaacctcttgtctctcctaaaagcatattatgctttgaatgcacaaccaagtgcagaagagctctcaaaaattgctgattcagtaaacctaccactggatgtagtaaaaaagtggtttgaaaagatgcaagctggacagatttcagtgcagtcttctgaaccatcttctcctgaaccaggcaaagtaaatatccctgccaagaacaatgatcagcctcaatctgcaaatgcaaatgaaccccaggacagcacagtaaatctacaaagtcctttgaagatgactaactccccagttttaccagtgggatcaaccaccaatggttccagaagtagtacaccatccccatcacctctaaacctttcctcatccagaaatacacagggttacttgtacacagctgagggtgcacaagaagagccacaagtagaacctcttgatctttcactaccaaagcaacagggagaattattagaaaggtcaactatcactagtgtttaccagaacagtgtttattctgtccaggaagaacccttgaacttgtcttgcgcaaaaaaggagccacaaaaggacagttgtgttacagactcagaaccagttgtaaatgtaatcccaccaagtgccaaccccataaatatcgctatacctacagtcactgcccagttacccacaatcgtggccattgctgaccagaacagtgttccatgcttaagagcgctagctgccaataagcaaacgattctgattccccaggtggcatacacctactcaactacggtcagccctgcagtccaagaaccacccttgaaagtgatccagccaaatggaaatcaggatgaaagacaagatactagctcagaaggagtatcaaatgtagaggatcagaatgactctgattctacaccgcccaaaaagaaaatgcggaagacagaaaatggaatgtatgcttgtgatttgtgtgacaagatattccaaaagagtagttcattattgagacataaatatgaacacacaggtaaaagacctcatgagtgtggaatctgtaaaaaggcatttaaacacaaacatcatttgattgaacacatgcgattacattctggagaaaagccctatcaatgtgacaaatgtggaaagcgcttctcacactctgggtcttattctcaacacatgaatcatcgctactcctactgtaagagagaagcggaagaacgtgacagcacagagcaggaagaggcagggcctgaaatcctctcgaatgagcacgtgggtgccagggcgtctccctcacagggcgactcggacgagagagagagtttgacaagggaagaggatgaagacagtgaaaaagaggaagaggaggaggataaagagatggaagaattgcaggaagaaaaagaatgtgaaaaaccacaaggggatgaggaagaggaggaggaggaggaagaagtggaagaagaagaggtagaagaggcagagaatgagggagaagaagcaaaaactgaaggtctgatgaaggatgacagggctgaaagtcaagcaagcagcttaggacaaaaagtaggcgagagtagtgagcaagtgtctgaagaaaagacaaatgaagcctaatcgtttttctagaaggaaaataaattctaattgataatgaatttcgttcaatattatccttgcttttcatggaaacacagtaacctgtatgctgtgattcctgttcactactgtgtgtgtgtgcgcgtgcattgattactatccatttctttagtcaacgctctccacttcctgatttctgctttaaggaaaactgtgaactttctgcttcatgtatcagttttaaagcatcccaggcaaagatcatctacagattctaggaattctctcccctgaaatcaaaacctggagacttttttttcttattttagttgagaagttcataaactgctcaaggattagttttccaggactctgcggaggaacggcaggaagaacctcagagagggcagaggtgacttcaaagtgctggggactccgtcctgagggtcacttggccctgagcccctgcgtgcccttgcggaagcccagaagcttcttcctgctgcacctcccgtttccgctgctgctgacgtttatgcatttcatgatggggtccaacaagaacacctgacttgggtgaagttgtgcaatattggaggctgactgtagggctgggcagctgggagacaggctcatggctcatggctcatggctcagggcggtgcctgccctgggccgggacccccctccccaccccccacctaggctttttgggttttgttcaaggaaggtaaagtgagaggtttaggtcagtgtttttaagtttttgttttttttttaaagcaaatcctgtatatgtatctacatgggagataggtagacactacttatttgttacattttgtactatacgtttgtgttccaggtttcagcttccctcgctcctgttgttaagaagcgtccctgtcagcacaggtgtgcattgaggaaggggccccagggccttcgctccctcagcactggggtggaggcggcaggaaggggcggcccttacctggcaggtctgggcgcacctttagcaggtggactccgtggggctccaccagccagaagcctctggaaggcaacgaaggcaatgctgctccctgagtccagtccccgcccccaaacccagcccaggtgccttcagctacttcggcttcttaaaccctgcagtgttaaacagaggcattgagaaaggggaaaggcgggtatttttaaaagccaaagattgacccaagttacttgagggtagggaggcgggcccagtgcaggaggctgcatccctggcctgctggtgcccaccgggggctgtgcctgtgccgggccgcaggaagctggctgcccccattcctgctgctgctgctgctgctgctctgtggctgtttcaaagactgggcgaaaggctgtccggagggcagaccaggtgccttgccgcagagaaaacaccaaagtctcctgttcgctcataaagaagtttttgggatgggagagaatccagaccatcttggggcagccaggcccttgccttcatttttacagaggtagcacaactgattccaacacaaaaccccttcccctttttaaaatgatttctgttctaatgccatagatcaaaggcctcagaaaccattgtgtgtttcctctttgaagcaatgacaagcactttactttcacggtggtttttgttttttcttattgctgtggaacctcttttggaggacgttaaaggcgtgttttacttgtttttttaagagtgtgtgatgtgtgttttgtagatttcttgacagtgctgtaatacagacggcaatgcaatagcctatttaaagaactacgtgatctgattgagatgtacatagtttttttttttaccataactgaattattttatctcttatgttatcatgagaaatgtatgccaaatgattagttgatgtatgttttttaatttaatatttaaataaaatatttggaagg

SEQ ID NO: 2 - DNA, 1702 bp, Homo sapiens
agactcaacaagagctccagcaaagactttcactgtagcttgacttgacctgagattaactagggaatcttgagaataaagatgagctctgaaaattgtttcgtagcagagaacagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagcccaaaatgcttgttctgaacatggtgctactcttgctgtcattgattctgaaaaggacatgaactttctaaaacgatacgcaggtagagaggaacactgggttggactgaaaaaggaacctggtcacccatggaagtggtcaaatggcaaagaatttaacaactggttcaacgttacagggtctgacaagtgtgtttttctgaaaaacacagaggtcagcagcatggaatgtgagaagaatttatactggatatgtaacaaaccttacaaataataaggaaacatgttcacttattgactattatagaatggaactcaaggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacaggttgaccaataaccatgcccaagaatgagaagaatgactatgcaacctttggatgcactttatattattttgaatccagaaataatgaaataactaggcgtggacttactatttattgctgaatgactaccaacagtgagagcccttcatgcatttgcactactggaaggagttagatgttggtactagatactgaatgtaaacaaaggaattatggctggtaacataggtttttagtctaattgaatcccttaaactcagggagcatttataaatggacaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcatttaaaatatttttatgtgcaataaaatgtatttgtttgtattttgtgttcagtacaattataagctgtttttatatatgtgaaataaaagtagaataaacacaaaaaaaaaaaaaaaaaaaaaaaaaaaa

SEQ ID NO: 3 - DNA, 6210 bp, Homo sapiens
gcaggaacagtgctagtattgctcgagcccgagggctggaggttaggggatgaaggtctgcttccacgctttgcactgaattagggctagaattggggatgggggtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttcttcggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacaggtgaggaactctagcgtactcttcctgggaatgtgggggctgggtgggaagcagccccggagatgcaggagcccagtacagaggatgaagccactgatggggctggctgcacatccgtaactgggagccctggctccaagcccattccatcccaactcagactctgagtctcaccctaagaagtactctcatagtttcttccctaagtttcttaccgcatgctttcagactgggctcttctttgttctcttgctgaggatcttattttaaatgcaagtcacacctattctgcaactgcaggtcagaaatggtttcacagtggggtgccaggaagcagggaagctgcaggagccagttctactggggtgggtgaatggaggtgatggcagacacttttactgaatgtcggtctttttttgtgattattctagttatctccagaagaagaagagaaaaggagaatccgaagggaaaggaataagatggctgcagccaaatgccgcaaccggaggagggagctgactgatacactccaagcggtaggtactctgtgggttgctcctttttaaaacttaagggaaagttggagattgagcataagggcccttgagtaagactgtgtcttatgctttcctttatccctctgtatacaggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaaccttgtgctcttttcattctggaagtcttgtaagtttctgaaaggtattattggagaccagtttgtcaagaagggtagctgctggagggggacacaccctctgtctgatcccttatcaaagaggacaaggaaactatagagctgattttagaatattttacaaatacatgccttccattggaatgctaagattttctactgcttctggggacgggaaaccgctgtgtaacagcttttgtgggaatacattttttctgtttcagtactcgcagggggaaatatttaaattttgttgtgctaatattaaattcagatgttttgatcttaaaggaaccctttaagcaaacagaacctagctttgtacagactattttaactttttattctcacaaaatcacgtggagggttattctacttcaaagatgagcaaattgaagaatggttagaataaacaactttcttgatattccgttatcggcattagaatcttcctgctcgttatcgtatccagcaggctgaactgcctcttgatacttggttaaaaaaaattttcaggccgggcgcggtggcccatgcctgtaatcctagcactttgggaggccgaggcaggcggatcacctgaggtcgggagttcgagaccagcctgaccaacatggagaaaccccgtctttactaaaaatacaaaattagcctggtgtggtggtgcatgcctgtaatcctagctacttgagaggctgagacaggaaaatcacttgaactcgggaggcggatgttgcagcgaactgagattgcgccattgcactccagcctgggcaacaagattgaaactctgtttaaaaaaaaaagttttcactaatgtgtacatttttttgtactcttttattctcgaaagggaaggagggctattgccctatcccttattaataaatgcattgtggtttctggtttctctaataccatatgcccttcattcagtttatagtgggcggaagtgggggagaaaaagttgctcagaaatcaaaagatatctcaaacagcacaaataatggctgatcgttctgcaaacaaaaagttacataatagctcaagaaggagaagtcaacatgactctgaacaagctttaacttagaaactttatcatcttaaggaagaacgtgacctttgtccaggacgtctctggtaatggggcacttacacacacatgcacacgtacaaaccacagggaaaggagaccgcccttctgcctctgctcgcgagtatcacgcaggcaccatgcactatgttttcacacacactgggtggaagaagagcttcagcgccagtcttctaatgctttggtgataatgaaaatcactgggtgcttatggggtgtcatattcaatcgagttaaaagttttaattcaaaatgacagttttactgaggttgatgttctcgtctatgatatctctgcccctcccataaaaatggacatttaaaagcaacttaccgctctttagatcactcctatatcacacaccacttggggtgctgtttctgctagacttgtgatgacagtggccttaggatccctgtttgctgttcaaagggcaaatattttatagcctttaaatatacctaaactaaatacagaattaatataactaacaaacacctggtctgaaataacaaggtgatctaccctggaaggaacccagctggtgggccaggagcggtggctcacacctgtaattccagcactttgggaggctgagacaggaggatcactggagtccaggagtttgagaccagcctgggcaacatggcaaaacccagtgtgcttctgttgtcccagctacactactcaggaggctgaggcaggagtatgacttgagcctgggagggggaggttgcagagaactgatattgcaccaccactgcactccagcctgggtgacacagcaaaaccctatctcaaaaaaaaaaaaaaaaaaaaggaacccagctggttcctgtaggtgtgcaataataacaaccagaggaagaaaaggaagacgatttcccagatgaagaagggcagctggaccttcggac

SEQ ID NO: 4 - DNA, 3622 bp, Homo sapiens
cccggggaggggaccggggaacagagggccgagaggcgtgcggcaggggggagggtaggagaaagaagggcccgactgtaggagggcagcggagcattacctcatcccgtgagcctccgcgggcccagagaagaatcttctagggtggagtctccatggtgacgggcgggcccgcccccctgagagcgacgcgagccaatgggaaggccttggggtgacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtggttcgggagtgtccagagagccttgtctccagccggccccgggaggagagccctgctgcccaggcgctgttgacagcggcggaaagcagcggtaccccacgcgcccgccgggggacgtcggcgagcggctgcagcagcaaagaactttcccggcggggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcacgccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggaggggttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttcggtctgacggcgcccccagtgtgcacgagtgggaaggacttggtcgcgccctcccttggcgtggagccagggagcggccgcctgcgggctgccccgctttgcggacgggctgtccccgcgcgaacggaacgttggactttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgctttcgttaactgtgtatgtacatatatatattttttaatttgattaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatcctagtctatctaggtggagttgaaagagttaagaatgctcgataaaatcactctcagtgcttcttactattaagcagtaaaaactgttctctattagacttagaaataaatgtacctgatgtacctgatgctatgtcaggcttcatactccacgctcccccagcgtatctatatggaattgcttaccaaaggctagtgcgatgtttcaggaggctggaggaaggggggttgcagtggagagggacagcccactgagaagtcaaacatttcaaagtttggattgcatcaagtggcatgtgctgtgaccatttataatgttagaaattttacaataggtgcttattctcaaagcaggaattggtggcagattttacaaaagatgtatccttccaatttggaatcttctctttgacaattcctagataaaaagatggcctttgtcttatgaatatttataacagcattctgtcacaataaatgtattcaaataccaataacagatcttgaattgcttccctttactacttttttgttcccaagttatatactgaagtttttatttttagttgctgaggtt

SEQ ID NO: 5 - DNA, 1080 bp, Homo sapiens
ccggggggcatgagggtccgagacttgttcttctgtcccttccaagacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctgcggagccttaaacaacgcagtgagacagacatctatcatcccattttacaggggaggatactgaggcacacagaggggagtcaccagccagaggatgtatagcctggacacagaggaagttggctagaggccggtcccttccttgggcccctctcattccctccccagaatggaggcaacgccagaatccagcaccggccccatttacccaactctgaacaaagcccttgcccccatgaaattgtttataaatcatccttttctccca

SEQ ID NO: 6 - DNA, 895 bp, Homo sapiens
agagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagatgtgcttttccagactgatccaactgcagagatggcagctgagtcattgcctttctcttcggacactgtcagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctat

SEQ ID NO: 7 - DNA, 2639 bp, Homo sapiens
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 8 - DNA, 1402 bp, Homo sapiens
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 9 - DNA, 3579 bp, Homo sapiens
cacgcgtccgcgagaaggaggactcgcaagcctcggcggcccggaaccggcctcggactgtcgacggaacctgaggccgcttgccctcccgccccatggagcggcccccggggctgcggccgggcgcgggcgggccctgggagatgcgggagcggctgggcaccggcggcttcgggaacgtctgtctgtaccagcatcgggaacttgatctcaaaatagcaattaagtcttgtcgcctagagctaagtaccaaaaacagagaacgatggtgccatgaaatccagattatgaagaagttgaaccatgccaatgttgtaaaggcctgtgatgttcctgaagaattgaatattttgattcatgatgtgcctcttctagcaatggaatactgttctggaggagatctccgaaagctgctcaacaaaccagaaaattgttgtggacttaaagaaagccagatactttctttactaagtgatatagggtctgggattcgatatttgcatgaaaacaaaattatacatcgagatctaaaacctgaaaacatagttcttcaggatgttggtggaaagataatacataaaataattgatctgggatatgccaaagatgttgatcaaggaagtctgtgtacatcttttgtgggaacactgcagtatctggccccagagctctttgagaataagccttacacagccactgttgattattggagctttgggaccatggtatttgaatgtattgctggatataggccttttttgcatcatctgcagccatttacctggcatgagaagattaagaagaaggatccaaagtgtatatttgcatgtgaagagatgtcaggagaagttcggtttagtagccatttacctcaaccaaatagcctttgtagtttaatagtagaacccatggaaaactggctacagttgatgttgaattgggaccctcagcagagaggaggacctgttgaccttactttgaagcagccaagatgttttgtattaatggatcacattttgaatttgaagatagtacacatcctaaatatgacttctgcaaagataatttcttttctgttaccacctgatgaaagtcttcattcactacagtctcgtattgagcgtgaaactggaataaatactggttctcaagaacttctttcagagacaggaatttctctggatcctcggaaaccagcctctcaatgtgttctagatggagttagaggctgtgatagctatatggtttatttgtttgataaaagtaaaactgtatatgaagggccatttgcttccagaagtttatctgattgtgtaaattatattgtacaggacagcaaaatacagcttccaattatacagctgcgtaaagtgtgggctgaagcagtgcactatgtgtctggactaaaagaagactatagcaggctctttcagggacaaagggcagcaatgttaagtcttcttagatataatgctaacttaacaaaaatgaagaacactttgatctcagcatcacaacaactgaaagctaaattggagttttttcacaaaagcattcagcttgacttggagagatacagcgagcagatgacgtatgggatatcttcagaaaaaatgctaaaagcatggaaagaaatggaagaaaaggccatccactatgctgaggttggtgtcattggatacctggaggatcagattatgtctttgcatgctgaaatcatggagctacagaagagcccctatggaagacgtcagggagacttgatggaatctctggaacagcgtgccattgatctatataagcagttaaaacacagaccttcagatcactcctacagtgacagcacagagatggtgaaaatcattgtgcacactgtgcagagtcaggaccgtgtgctcaaggagctgtttggtcatttgagcaagttgttgggctgtaagcagaagattattgatctactccctaaggtggaagtggccctcagtaatatcaaagaagctgacaatactgtcatgttcatgcagggaaaaaggcagaaagaaatatggcatctccttaaaattgcctgtacacagagttctgcccggtcccttgtaggatccagtctagaaggtgcagtaacccctcagacatcagcatggctgcccccgacttcagcagaacatgatcattctctgtcatgtgtggtaactcctcaagatggggagacttcagcacaaatgatagaagaaaatttgaactgccttggccatttaagcactattattcatgaggcaaatgaggaacagggcaatagtatgatgaatcttgattggagttggttaacagaatgagttgtcacttgttcactgtccccaaacctatggaagttgttgctatacatgttggaaatgtgtttttcccccatgaaaccattcttcagacatcagtcaatggaagaaatggctatgaacagaaactacatttctactatgatcagaagaacatgattttacaagtataacagttttgagtaattcaagcctctaaacagacaggaatttagaaaaagtcaatgtacttgtttgaatatttgttttaataccacagctatttagaagcatcatcacgacacatttgccttcagtcttggtaaaacattacttatttaactgattaaaaataccttctatgtattagtgtcaacttttaacttttgggcgtaagacaaagtgtagttttgtatacagagaagaaaacctcaagtaataggcattttaagtaaaagtctacctgtgtttttttctaaaaaggctgctcacaagttctatttcttgaagaataaattctacctccttgtgttgcactgaacaggttctcttcctggcatcataaggagttggtgtaatcattttaaattccactgaaaatttaacagtatccccttctcatcgaagggattgtgtatctgtgcttctaatattagttggctttcataaatcatgttgttgtgtgtatatgtatttaagatgtacatttaataatatcaaagagaagatgcctgttaatttataatgtatttgaaaattacatgttttttcatttgtaaaaatgagtcatttgtttaaacaatctttcatgtcttgtcatacaaatttataaaggtctgcactcctttatctgtaattgtaattccaaaatccaaaaagctctgaaaacaaggtttccataagcttggtgacaaaattcatttgcttgcaatctaatctgaactgaccttgaatctttttatcccatttagtgtgaatattcctttattttgctgcttgatgatgagagggagggctgctgccacagactgtggtgagggctggttaatgtagtatggtatatgcacaaaactacttttctaaaatctaaaatttcataattctgaaacaacttgccccaagggtttcagagaaaggactgtggacctctatcatctgctaagtaatttagaagatattatttgtcttaaaaaatgtgaaatgcttttatattctaatagtttttcactttgtgtattaaatggtttttaaattaaaaaaaaaaaaaaaa

SEQ ID NO: 10 - DNA, 1933 bp, Homo sapiens
cctggccggtgcttcagttagatcaaaccattgctgaaactgaagaggacatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagacctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgaaaaccatcgatgaaggaatatctcttctcagaaggaaagaataaccaacaccctgaggttgtgtgtggaaggtgatctggctctggacaggcactatctgggttttggggggacgctataggatgtggggaagttaggaactggtgtcttcaggggccacaccaaccttctgaggagctgttgaggtacctccaaggaccggcctttgcacctccatggaaacgaagcaccatcattcccgttgaacgtcacatctttaacccactaactggctaattagcatggccacatctgagccccgaatctgacattagatgagagaacagggctgaagctgtgtcctcatgagggctggatgctctcgttgaccctcacaggagcatctcctcaactctgagtgttaagcgttgagccaccaagctggtggctctgtgtgctctgatccgagctcaggggggtggttttcccatctcaggtgtgttgcagtgtctgctggagacattgaggcaggcactgccaaaacatcaacctgccagctggccttgtgaggagctggaaacacatgttccccttgggggtggtggatgaacaaagagaaagagggtttggaagccagatctatgccacaagaaccccctttacccccatgaccaacatcgcagacacatgtgctggccacctgctgagccccaagtggaacgagacaagcagcccttagcccttcccctctgcagcttccaggctggcgtgcagcatcagcatccctagaaagccatgtgcagccaccagtccattgggcaggcagatgttcctaataaagcttctgttcc

SEQ ID NO: 11 - DNA, 3429 bp, Homo sapiens
ggctggaagccggaagcgagcaaagtggagccgactcgaactccaccggcacgagggcggaaaagaaagcctcagaacgttcgctcgctgcgtccccagccggggccgagccctccgcgacgccacccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 12 - DNA, 1693 bp, Homo sapiens
tcgcgcctctggattccagcctggcgaccgaacgagactgctccatctccaaaaaaaaaaaaaaaggcctgtgagggatcctgtggctaaagtgagcccctctccaggtgccacatgcctcgacatgtgcctgcagcccgggatctcacccacccccactcacgactcacacactcacaacgtgcagttgggcgcctaggattgtgcatgttcaagtctccacccactccctttgttaatcgtcggaatttccagcccgctgctgccaaccgctccccagctgcgggaggaggagtagaaggacccgcccaattttcaggagcacataaattacctctgccggcagccgaccctcacttggccttacactccgctcggctcaccatgtgtcactctcgcagctgccacccgaccatgaccatcctgcaggccccgaccccggccccctccaccatcccgggaccccggcggggctccggtcctgagatcttcaccttcgaccctctcccggagcccgcagcggcccctgccgggcgccccagcgcctctcgcgggcaccgaaagcgcagccgcagggtctctctaccctcgagtggtgagtatcgccgaagtgggcattcgcggtgtgcgctgccctggagtcactggggaacgacccgactccagagcctcgacctgacctgtctcctgttttgtctccccttagtccggcgccagctgccagtcgaggaaccgaacccagccaaaaggcttctctttctgctgctcaccatcgtcttctgccagatcctgatggctgaagagggtgtgccggcgcccctgcctccagaggacgcccctaacgccgcatccctggcgcccacccctgtgtcccccgtcctcgagccctttaatctgacttcggagccctcggactacgctctggacctcagcactttcctccagcaacacccggccgccttctaactgtgactccccgcactccccaaaaagaatccgaaaaaccacaaagaaacaccaggcgtacctggtgcgcgagagcgtatccccaactgggacttccgaggcaacttgaactcagaacactacagcggagacgccacccggtgcttgaggcgggaccgaggcgcacagagaccgaggcgcatagagaccgaggcacagcccagctgggctaggccggtgggaaggagagcgtcgttaatttatttcttattgctcctaattaatatttatatgtatttatgtacgtcctcctaggtgatggagatgtgtacgtaatatttattttaacttatgcaagggtgtgagatgttccctctgctgtaaatgcaggtctcttggtatttattgagctttgtgggactggtggaagcaggacacctggaactgcggcaaagtaggagaagaaatggggaggactcgggtgggggaggacgtcccggctgggatgaagtctggtggtgggtcgtaagtttaggaggtgactgcatcctccagcatctcaactccgtctgtctactgtgtgagacttcggcggaccattaggaatgagatccgtgagatccttccatcttcttgaagtcgcctttagggtggctgcgaggtagagggttgggggttggtgggctgtcacggagcgactgtcgagatcgcctagtatgttctgtgaacacaaataaaattgatttac

SEQ ID NO: 13 - DNA, 2934 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaacacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcaccttactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtggtttatacctcgcagatacaagaatctttatgaatatacaattctttttccttctacagcttagctccgtcttttcaaccacgaacatttaaaacccgacctactagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcaaaaaaaaaaaaaaaaaaa

SEQ ID NO: 14 - DNA, 1514 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggtacctggagtttaatacagtgagctctagcaagggaggcgctgccttttgtttctagaatattatgttcctcaaggtccattattttgtattcttttccaagctccttattggaaggtatttttttaaatttagaattaaaaattatttagaaaaaaaaaaaaaaaaaaaaaaaaaaaaaa

SEQ ID NO: 15 - DNA, 7635 bp, Homo sapiens
tgtccatcctgtaacaattgaaaatttctggctgggcgtggtggctcacgcctgtaatcccagcactttgagaggccgaggcaggtggatcacctcaggtcaggtgttcaagaccagcctggccaacatggtgaaaccccgtctctactaaaaaaaaataataataataatacaaaaattagccgggggtggtggtaggcacctgtaatcccagatactcgggaggctgaggcaggagactcacttgaacctgggaggcggaggttgcaatgagctgagatcgcgccactgtactccagcctggatgacagagcaggactccatctcaaaaagaaaggaaaagaaaagaaaaaatattaaatgtgtacgctctttgactcagctgtattacttcaaggagttgatatcaccaaaattgcctaagtgctcaaaggtgtttgtagttaaacaacaggagattgataaattatgttatatacatgtgatgctatgttttaaagaggtactgatatgataaaagatgtacgtggcataaaattaaatgtactttattaagtacttttccaagtgtttacggaatgagtgcatttttgaaaaaaaaaaagtgtattcgaacttttaaaaaagctttaaaagctttatacaatgaacgattgagtgattataagagctggcgggggaatgttaagaggatgatagggagctaagtttaacagaacaattcacctctttatcttgtgacacctacgagcgcatcaattctgtaattgaaaaataaagtgcatatttgcagcagctgtactctcttcaggctgcaaggaggcttttcctcccggtaggcttgatttgcatttcactttcactttcgtggctggaaactttctacccacgtagtgggaggctgaggagccaccataaagctggggcttgacgagccgggaccgggacccgatctccacatatgcccggacttgttctgcggccgggttcaggagtcaaagaggcggggagacctgcgcgacgctgccccgccctgcgcccgcttcctccaatgtatgctctagggggcgggcctcgcggggagcatggacacgattggccctaaagtcttccccgcaaggccgtgggctggacagcgtggtgacgtcgcaacgcggcgcagggtgagagcgcgcgcttgcggacgcggcggcattaaacggttgcaggcgtagcagagtggtcgttgtctttctaggtctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccaggtgagcctcgcggccccgggaagccggccccggcccgcctgcacctccggtgcttggcgggagcgctttcgagcctagccctcattggctggcgtgggccatccgcgccttctcattggcctgccacgcagtggggtggggcccagctgagcgcgcggttcggaaaagcccgcgctggctgctgcgcgaacctgctttttcgcgccaaagtcacaaagcgggtggtggcgggaaaatcaagggtttttccgcagtgccaggaacactgttccagggctctttgctcactaaatcctgttggccttgaatggacgctttagctgtggctttcttgtttctgagacggtctcggtgtgttgcccgggctggtctccaacttctgggctcaagcgatcctcccggctcagtcgcggttgactttaaatgctttataatgcccttgcgagaaatgtggcagcctgtcatcctacttagtggcaggagattgtttctatccagaagggacactgctggtggtattttagtataaatactgccagatgcgtcccaaaacgtctgcattaataatggcatcctccagcagtccgcttaccctccaccagttctgagacggcctgatgggtgagagtggtaaccccttctaaccgcgttcgaaatacagcccttcagcagacggcgttgattttaaagcatgtgtctcctgtcttctagtatgtccaaaatactaaaatgcgccggcaatgaagatatcattacactaagggccgaagataacgcggataccttggcgctagtatttgaagcaccaagtaagtcgtacctttttaccgagtcacgaagctacagaaaatcaaaactctgtgtcagtatttctcaaaagcatctgctttcttttggtagaccaggagaaagtttcagactatgaaatgaagttgatggatttagatgttgaacaacttggaattccagtgagtatcagtttctcattgtagagagtgctgtacacaggcacgatagttatgtcatagaatgtttgtttatttttacagacagggtcttggctctgttgcccaggctggagtgcagtagtgccatatagctctctctaacctgggattcctgggctcaagcagtcctcttgccttagtctcctaagtggctaggaaggactacgggcctgtcccaccacacctggctaattttttcatttttgtgtgtgggcgtgggggcagtcttgcccaggctggtctggaactcctggcctcaagtgatcctcctccgtcaagatatgttaatataatttaagcctacttcataacaactttttctagaaatatatctactggtgcatgtttcaaagagagattttagtatttggatagttgttcaccacaagtctaaaactcacaggttaaatttattgtttactgccagttgtctatttgcattaacttccatgaacctcttttaaaatttgtttccttctagaatgcttgctttttattaagtggattttaaagctgacttggaagaaatttatccaggttaggtttataaacaccaaaggagagaagaaatgtttgaatgttgaaaatgcctaatatattctcttgctttcttttagaaagtagttaggcctgcttgcgccatcatgatttctgtgccatactctaatgttctcttactttatccctggaggatgaggaggaggaggctcttgttccctggatggtgcatttaatagccatttatttttttgagtggagtttgttaagaaattacgcaagtcatattttaaagtaatcagaaaatattgattctgagttgtttaggtgttgccttttaagaaagtgagggtgccaaatcattaaatttctaacaattaacttttggaaaatttttgttcttaataggaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgtaagtagtttttaagtaaaaagaaaatagtttgaagagaattataatactgcttattaggttaattgctaaaattaaaagtagacagaattggatcccaagtaatttctgaaaattgagatactgttgaaatctgcgaatgatttataagtgtcatccaatttagaattatatttgcaagaagggaatacaaattcagcacgtgtacataccacagaacagtggtttatggatcaagtccacaccggctcttaagggtaggattgggaagttaggcgtataacctttagctttctggagatacttactctcttaccaaataattgagcataggacagcagctcaatagaaggatatggttaggagtaaaagtctacctgtttggagcacttatgtaatcctaatattagcttacatgttgtgggtccaattggtagcccattttaaaggtggagaagcaggctgagcaaccttaagtgacaatttagccaaagtcacaggctgtaggaatcaaaggttaaacaggaaggagacttctcactaaggctagaaagcagactccatgcaactttgagagtacctagagagcccttgtttaaccaaaatagaaagaacatggcaaaaccccatctctactaaaaatataaaaattagccgggtgtggtggcgcacacctgtaatcccagctacttggaatgctgagatgggagaatgacttgaaccgaggaggcggaggttgcagtgagcccagatcatgccactcccctccagcctgggtgacagagcaagattccatcttaaacaaacaaaaaaaactcgctaacctgggcataaattaaaactttgtaaatcaaggacaaaggtcctaacctcataaccttgttaggattaaatacggtagcattaaagagcttagcatatgctgtgtgtggcatattataagcttacaataaatactatatattgctctcttgtcccttgaatgggtagtcaacatttagtttaaataaaggtaaaattcagttgaaaggtttttttttaaattaataaagtctaggagctgattctttatctgtttcctgaatcacatttccactcctgccaacctcgtttttttctttttctgtttttctttgtttttgagacagggtcttgctctgtccacccaggctggagtgcggtggtgcagtcggttcactacagcctcaaactccagggcttaagtgatcctcctgcctcagtttcccaagtagccgggactacaggtgtgtgccaacacacctagcctggtttcccctaatttcattttcccccttccattacaatctatttgttgaagaaattagatcatttattaagttttccagagtttggattttgctgattgcattcctgtgtatacataaattcctctaccctgtgtgtcctacagactggtagctatagccctggagccttgatattcagggtgttttgtttcgggggtgagagagcaagaatataggtggtggtgtgtgcctctagtaggaggcacagggtgtctggatgtgtttgcaatgttagcagctataatagtcattgtctagatccattaagtcattaattagagtttgcagagctgaaattaatacgttttattcacttattggctgcttattagaaaaacttccataaagaaaagctttcccatattatataatttggttatctaaattatagctataggaaagacaagctagataatcgagtcttttgctttatgtatcagtcttcaaaattttcatagctccctccaaagtgaccaatacaagtgtttgtgggtttttataaatatataatgagctaatagattgcaactttcttgatgtttttcaatgatgaatcttttgttttgtaggttaccatagagatgaatgaaccagttcaactaacttttgcactgaggtacctgaacttctttacaaaagccactccactctcttcaacggtgacactcagtatgtctgcagatgtaccccttggtaagataataaatttgaaccttgttttgtaggtagtcatatgtgatacatactcctcagtaattaaccatcttcctgtctttcagttgtagagtataaaattgcggatatgggacacttaaaatactacttggctcccaagatcgaggatgaagaaggatcttaggcattcttaaaattcaagaaaataaaactaagctctttgagaactgcttctaagatgccagcatatactgaagtcttttctgtcaccaaatttgtacctctaagtacatatgtagatattgttttctgtaaataacctatttttttctctattctctgcaatttgtttaaagaataaagtccaaagtcagatctggtctagttaacctagaagtatttttgtctcttagaaatacttgtgatttttataatacaaaagggtcttgactctaaatgcagttttaagaattgtttttgaatttaaataaagttacttgaatttcaaacatcacagggcagtgtcttcattttgaccaggactgttggaagtatcctactgaattcccagctacagctaccctttcgtttaaatggtttttcagtttagagcagcctgatggagatcaaaaggatttcaaatacagtaggttactagatattttaggggattcacttgtcaaacttgttattccaatttgagctcaaggaacaagtgagagagtagtacaaatcctgtgtatttcattttacttggcaaaaattaatactcttcagaagcttaatgtacccaaagtcgtgtgggctttataagatgttaatggagtgtttctttttagcctggggaatcttacaagagtcttggatgtttcagtagagctgtaatggggcccaaggagtatggttgtacaaaatttctctagtataagtacactttgcaacttcactcaaaggatgaagatgtcttaaattccacggatgttggatccaaccaagaaagtgaaaaagccctgaagttgtgaagaaagacttaaaaacattcccactaatagctacaagttcactgtgtgctgtgtattaagaattttttgccaggcacggtgcctcacacctgtaatcccagcactttgggaggtaggcggatcaactaaagtcaggagttcgagaccagcctggtcaacatggtgaaaccccatttctactaaaaatgcaaaaaaaaaattagccaggcatggtggcacatgcctgtagtcccagctacttgggaggctgaggcagaagaatcacttaaacccaggaggtggaggttgcagtgagccaagatcatgccactgcactccagcctggtcaacagagtgagactccatctcaaaaaaaattattggctggtttaatagtagtaggttatcagaacttactaacattagtatcactaaagtaggtatacaacccctgctgctcaatttggctttagaaaaaacatttttaaaaactgctttatcataatccttttttccagtgcatttttgtgtttggtagattaaatgactgatttctttcttaaattacttggactgagttctagttactggacatgagaattaaccatgtccagtagttaagttcattttcctacagtgtgcctgtgagtggagaaggaaataggctgcaaaagagcccgatggaactttggggtgatggatatatttccatctagattggtgatggttctaggaccgtatgtattttcagagctcatctatttgtacttgtgtataaatacttcaataaacctaactttttaaaaagcttcatttgccgcaaaatgtcccttgtgggcaggccttttacctggacaagccctaaagataagcacaagtttgtggggctattcaagggaagtatagtggggtgctgacagcacagacaagtgtgctattcaagggaagt

SEQ ID NO: 16 - DNA, 1927 bp, Homo sapiens
gtggagtttgtgacatacgaggtgacacccctcgagtcacttcccttcaactccagctggagcgcctgcttggctttgggttcgttctgcagccttcgccccgctcctagcctcagggccggactccagcgcagagcccagcccagcgcagcctgccagcagccacccagccgcccagccgcccagccccgcacgaaacccggccagagcttcctagcagcccgagccatgaacaccgaaatgtatcagacccccatggaggtggcggtctaccagctgcacaatttctccatctccttcttctcttctctgcttggaggggatgtggtttccgttaagctggacaacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagcactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgctccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccataggcctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaat

SEQ ID NO: 17 - DNA, 789 bp, Homo sapiens
gccaccgcccgcagctgaagcacatccgcagcccggcgcgactccgatcgccgcagttgccctctggcgccatgtccgagaacggagcgcccgggatgcaggaggagagcctgcagggctcctgggtagaactgcacttcagcaataatgggaacgggggcagcgttccagcctcggtttctatttataatggagacatggaaaaaatactgctggacgcacagcatgagtctggacggagtagctccaagagctctcactgtgacagcccacctcgctcgcagacaccacaagataccaacagggcttctgaaacagatacccatagcattggagagaaaaacagctcacagtctgaggaagatgatattgaaagaaggaaagaagttgaaagcatcttgaagaaaaactcagattggatatgggattggtcaagtcggccggaaaatattccccccaaggagttcctctttaaacacccgaagcgcacggccaccctcagcatgaggaacacgagcgtcatgaagaaagggggcatattctctgcagaatttctgaaagttttccttccatctctgctgctctctcatttgctggccatcggattggggatctatattggaaggcgtctgacaacctccaccagcaccttttgatgaagaactggagtctgacttggttcgttagtggattacttctgagcttgcaacatagctcactgaagagctgttagatcctgggccttcgtggctcgagagactagaatcgcagatacgaaaaccccgcagc

SEQ ID NO: 18 - DNA, 3900 bp, Homo sapiens
cgatggccctgcccagtcccagacagaacctactacgtgcggcggcagctggggcgggaaggcgggcgctgggggcgctgcggccgctgcagcgcagggtccacctggtcggctgcacctgtggaggaggaggtggatttcaggcttcccgtagactggaagaatcggctcaaaaccgcttgcctcgcaggggctgagctggaggcagcgaggccgcccgacgcaggcttccggcgagacatggcagggcaaggatggcagcccggcggcagggcccggcgaggagcgcgaacccgcggccgcagttcccaggcgtctgcgggcgcgagcacgccgcgaccctgcgtgcgccggggcgggggggcggggcctcgcctgcacaaatagggacgagggggcggggcggccacaatttcgcgccaaacttgaccgcgcgttctgctgtaacgagcgggctcggaggtcctcccgctgctgtcatggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaatatgaaggtgttttctagtttaagttgttccccctccctctgaaaaaagtatgtatttttacattagaaaaggttttttgttgactttagatctataattatttctaagcaacttgtttttattccccactactcttctctctatcagataccatttatgagacattcttgctataactaagtgcttctccaagaccccaactgagtccccagcacctgctacagtgagctgccattccacacccatcacatgtggcactcttgccagtccttgacattgtcgggcttttcacatgttggtaatatttattaaagatgaagatccacatacccttcaactgagcagtttcactagtggaaataccaaaagcttcctacgtgtatatccagaggtttgtagataaatgttgccaccttgtttgtaacagtgaaaaattgaaaacaacctggaagtccagtgatgggaaaatgagtatgtttctgtcttagattggggaacccaaagcagattgcaagactgaaatttcagtgaaagcagtgtatttgctaggtcataccagaaatcatcaattgaggtacggagaaactgaactgagaaggtaagaaaagcaatttaaagtcagcgagcaggttctcattgataacaagctccatactgctgagatacagggaaatggaggggggaaagctggagtattgatcccgcccccctccttggttgtcagctccctgtcctgtgtgtgggcggaacatagtccagctgctctatagcaagtctcaggtgtttgcagtaagaagctgctggcatgcacgggaacagtgaatgccaaacacttaaagcaattcgatgtttaagtatgtaagttcttttttttttagacagcgtttcgctcttgttgcccaggctagcatgcaatggtgtgacctcggcttactgcaacctccgccttcccagattcaagcgattctcctgcctcaggctcccaagtagctaggaccaggtgcgcgccaccacgcccggctaatttttgtattttgtatttttagtagagatggggtttcaccatgttggtcaggctagtctcgaactcgtgaccgcaagcgattcacccacctcagcctcccaaagtgctgggattaccggcttgagccaccacacccggcacatcttcattctttttatgtagtaaaaagtataaggccacacatggtttatttgaagtattttataatttaaaaaaatacagaagcaggaaaaccaattataagttcaagtgagggatgatggttgcttgaaccaaagggttgcatgtagtaagaaattgtgatttaagatatattttaaagttataagtagcaggatattctgatggagtttgactttggttttgggcccagggagtttcagatgcctttgagaaatgaatgaagtagagagaaaataaaagaaaaaccagccaggcacagtggctcacacctgtaatcccagcgctttgggaggctaaggcaggcagatcacttgagaccagcttgggcaacatggcaaagccccatctctacaaaaaacacaaaaattagctgggcattgtggcgcacacctgtattcccatctagtcaggaagctgagatggaagaattaattgagcccacgagttcaaggctgcagtgagtcgtgattgtgccactgcactccagccggggtgacagaagagaccttgtctcgaaaacgaatctgaaaacaatggaaccatgccttcataattctagaaagttattttcaactgataaatctatattcacccaaataatcaagggtgaaggtaaaataatacatttttagacaagcaaagactcaggggttacctccatgtgccctttttagggaagctgttggagaaaatactccagcaaaatgaaggagtacacaaaccagagaatgacatgaatccagcaaataggatccaacacaggcaatattccagctatggagctagctttaaaaaggaacagtaaaaatattaatcggttagctgggtggaatggcccatgcctgtagtcccagctactcaggaggctcagcagcaggacgacttgagcccaagagttccagaccagcctggccaccttagtgagatcccttctcttaaaaataataacttattgccagatttggggcatttggaaagaagttcattgaagataaagcaaaagtaaaaaaaaaaaaaaaaaaaacaaggggaaagggttggttaggcaatcattctagggcagaaagaagtacaggataggaagagcataatacactgtttttctcaacaaggagcagtatgtacacagtcataatgatgtgactgcttagcccctaaatatggtaactactctgggacaatatgggaggaaaagtgaagattgtgatggtgtaagagctaatcctcatctgtcatatccagaaatcactatataatatataataatgaaatgactaagttatgtgaggaaaaaaacagaagacattgctaaaagagttaaaagtcattgctctggagaattaggagggatggggcaggggactgttaggatgcattataaactgaaaagcctttttaaaattttatgtattaatatatgcattcacttgaaaaactaaaaaaaaacaataatttggaaaaacccatgaaggtaactaacggaaggaaaaactaagagaatgaaaagtatttgcctctggaaagaacaactggcaggactgttgttttcattgtaagacttttggagccatttaattgtacttaaccattttcatctatttctttaataagaacaattccatcttaataaagagttacacttgttaata

SEQ ID NO: 19 - DNA, 2881 bp, Homo sapiens
acagaagtgctagaagccagtgctcgtgaactaaggagaaaaagaacagacaagggaacagcctggacatggcatcagagatccacatgacaggcccaatgtgcctcattgagaacactaatgggcgactgatggcgaatccagaagctctgaagatcctttctgccattacacagcctatggtggtggtggcaattgtgggcctctaccgcacaggcaaatcctacctgatgaacaagctggctggaaagaaaaagggcttctctctgggctccacggtgcagtctcacactaaaggaatctggatgtggtgtgtgccccaccccaagaagccaggccacatcctagttctgctggacaccgagggtctgggagatgtagagaagggtgacaaccagaatgactcctggatcttcgccctggccgtcctcctgagcagcaccttcgtgtacaatagcataggaaccatcaaccagcaggctatggaccaactgtactatgtgacagagctgacacatagaatccgatcaaaatcctcacctgatgagaatgagaatgaggttgaggattcagctgactttgtgagcttcttcccagactttgtgtggacactgagagatttctccctggacttggaagcagatggacaacccctcacaccagatgagtacctgacatactccctgaagctgaagaaaggtaccagtcaaaaagatgaaacttttaacctgcccagactctgtatccggaaattcttcccaaagaaaaaatgctttgtctttgatcggcccgttcaccgcaggaagcttgcccagctcgagaaactacaagatgaagagctggaccccgaatttgtgcaacaagtagcagacttctgttcctacatctttagtaattccaaaactaaaactctttcaggaggcatccaggtcaacgggcctcgtctagagagcctggtgctgacctacgtcaatgccatcagcagtggggatctgccgtgcatggagaacgcagtcctggccttggcccagatagagaactcagctgcagtgcaaaaggctattgcccactatgaacagcagatgggccagaaggtgcagctgcccacagaaagcctccaggagctgctggacctgcacagggacagtgagagagaggccattgaagtcttcatcaggagttccttcaaagatgtggaccatctatttcaaaaggagttagcggcccagctagaaaaaaagcgggatgacttttgtaaacagaatcaggaagcatcatcagatcgttgctcaggtttacttcaggtcattttcagtcctctagaagaagaagtgaaggcgggaatttattcgaaaccagggggctatcgtctctttgttcagaagctacaagacctgaagaaaaagtactatgaggaaccgaggaaggggatacaggctgaagagattctgcagacatacttgaaatccaaggagtctatgactgatgcaattctccagacagaccagactctcacagaaaaagaaaaggagattgaagtggaacgtgtgaaagctgagtctgcacaggcttcagcaaaaatgttgcaggaaatgcaaagaaagaatgagcagatgatggaacagaaggagaggagttatcaggaacacttgaaacaactgactgagaagatggagaacgacagggtccagttgctgaaagagcaagagaggaccctcgctcttaaacttcaggaacaggagcaactactaaaagagggatttcaaaaagaaagcagaataatgaaaaatgagatacaggatctccagacgaaaatgagacgacgaaaggcatgtaccataagctaaagaccagagccttcctgtcacccctaaccaaggcataattgaaacaattttagaatttggaacaagcgtcactacatttgataataattagatcttgcatcataacaccaaaagtttataaaggcatgtggtacaatgatcaaaatcatgttttttcttaaaaaaaaaaaaaagactgtaaattgtgcaacaaagatgcatttacctctgtatcaactcaggaaatctcataagctggtaccactcaggagaagtttattcttccagatgaccagcagtagacaaatggatactgagcagagtcttaggtaaaagtcttgggaaatatttgggcattggtctggccaagtctacaatgtcccaatatcaaggacaaccaccctagcttcttagtgaagacaatgtacagttatccattagatcaagactacacggtctatgagcaataatgtgatttctggacattgcccatgtataatcctcactgatgatttcaagctaaagcaaaccaccttatacagagatctagaatctctttatgttctccagaggaaggtggaagaaaccatgggcaggagtaggaattgagtgataaacaattgggctaatgaagaaaacttctcttattgttcagttcatccagattataacttcaatgggacactttagaccattagacaattgacactggattaaacaaattcacataatgccaaatacacaatgtatttatagcaacgtataatttgcaaagatggactttaaaagatgctgtgtaactaaactgaaataattcaattacttattatttagaatgttaaagcttatgatagtcttttctaattcttaacactcatacttgaaatctttccgagtttccccagaagagaatatgggattttttttgacatttttgacccatttaataatgctcttgtgtttacctagtatatgtagactttgtcttatgtgtcaaaagtcctaggaaagtggttgatgtttcttatagcaattaaaaattatttttgaactga

SEQ ID NO: 20 - DNA, 647 bp, Homo sapiens
gctcactgagcaccgtcccagcatccggacaccacagcggcccttcgctccacgcagaaaaccacacttctcataccttcactcaacacttccttccccaaagccagaagatgcacaaggaggaacatgaggtggctgtgctgggggcaccccccagcaccatccttccaaggtccaccgtgattaacatccacagcgagacctccgtgcccgaccatgtcgtctggtccctgttcaacaccctcttcttgaactggtgctgtctgggcttcatagcattcgcctactccgtaaagtctagggacaggaagatggttggcgacgtgaccggggcccaggcctatgcctccaccgccaagtgcctgaacatctgggccctgattctgggcatcctcatgaccattggattcatcctgttactggtattcggctctgtaacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtga

SEQ ID NO: 21 - DNA, 1826 bp, Homo sapiens
acagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcagtatgtgaaagccaggtcccccagagccaatctgccccctctctatgctcttgaacttctaaccatctatgcctgggaaatgggtactgaagaagacgagaatttcatgttggacgaaggcttcaccactgtgatggacctgctcctggagtatgaagtcatctgtatctactggaccaagtactacacactccacaatgcaatcattgaggattgtgtcagaaaacagctcaaaaaagagaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaa

SEQ ID NO: 22 - DNA, 1584 bp, Homo sapiens
acagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaa

SEQ ID NO: 23 - DNA, 3490 bp, Homo sapiens
cgccagagtccggagcagccgccgcccgaccgcgccgagctcagttcgctgtccgcgccggctcccaccccggcccgaccccgacccggcccggtcaggccccatactcagtagccacgatggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgacctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctaggcggcctggccccagtgacggccccctctttgcaggcttttcctcctctctagaacctccttctgttggaggccctcccatctccccgctgaaacctgcgctccttttttggggggatcctttgctgctgagcttccccaagcacggtgtgccctggcctgccttcttcttgtgtctttggtggggatggggaggcctattcctgctggccccttctgggggtggtgggcaggtgacacggagtggcttgagcttctggggatgcaggtccaccgagcccctgacccctgtctgtccccgctcccctaacaggtgcggttcctcatctgagaggctctccgtgcaggcgatggggcaagacagaaaagtgcctgagctggggaagccggggtgtaacttcctgctgcaccctgcgcctccagaggtcctccgtagggtctttcttgggatagtgttctgctcctgcttttctgtcctgggcatgggtccagggcctgacaccccctccccgcccctgtggccctggccactaaagcttcagactcaagtacccattctgttttcccccagcaacgcccctccaaacctccagcctccctgtctccagctgcctgggcccggaagggctttggttccttctctgggtctgattttctcactgaactccaccgaccaactgccctaagcccccagggcctccagggcccaggttcgagacccaaacccccaaaatccaaaacttctcttgaaaagttcagggaccgtccaggggagatggggaggagatatggagtgagtcacctgctccagaagatgccagcttctctctccagggtgcttagttggctttgcccacccctcactccccagggagctccggggacagcttcctcacacccctgtcccacccacacagctgccctagctgaccccgagaagtgctcttggctgacccctctggtgtgtggtgaggggctttctcttccccttcctgtttcagacccccccatttcccgcacatggtgtggggggctgggggaggtccaagcagagtgttttattattatcgctttatgtttttggttattggtttttttgtatagaccaaagcaaagaaaataaaaataacacag

SEQ ID NO: 24 - DNA, 3192 bp, Homo sapiens
cgccagagtccggagcagccgccgcccgaccgcgccgagctcagttcgctgtccgcgccggctcccaccccggcccgaccccgacccggcccggtcaggccccatactcagtagccacgatggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgacctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctaggcggcctggccccagtgcggttcctcatctgagaggctctccgtgcaggcgatggggcaagacagaaaagtgcctgagctggggaagccggggtgtaacttcctgctgcaccctgcgcctccagaggtcctccgtagggtctttcttgggatagtgttctgctcctgcttttctgtcctgggcatgggtccagggcctgacaccccctccccgcccctgtggccctggccactaaagcttcagactcaagtacccattctgttttcccccagcaacgcccctccaaacctccagcctccctgtctccagctgcctgggcccggaagggctttggttccttctctgggtctgattttctcactgaactccaccgaccaactgccctaagcccccagggcctccagggcccaggttcgagacccaaacccccaaaatccaaaacttctcttgaaaagttcagggaccgtccaggggagatggggaggagatatggagtgagtcacctgctccagaagatgccagcttctctctccagggtgcttagttggctttgcccacccctcactccccagggagctccggggacagcttcctcacacccctgtcccacccacacagctgccctagctgaccccgagaagtgctcttggctgacccctctggtgtgtggtgaggggctttctcttccccttcctgtttcagacccccccatttcccgcacatggtgtggggggctgggggaggtccaagcagagtgttttattattatcgctttatgtttttggttattggtttttttgtatagaccaaagcaaagaaaataaaaataacacag

SEQ ID NO: 25 - DNA, 860 bp, Homo sapiens
gactctcactgtcattgcagaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattcttttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaagtgatgtacaacc

SEQ ID NO: 26 - DNA, 4157 bp, Homo sapiens
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttcta

SEQ ID NO: 27 - DNA, 2762 bp, Homo sapiens
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagtgtaagtgaacacagaagagtgacatgtttacaaacctcaagccagccttgctcctggctggggcctgttgaagatgcttgtattttacttttccattgtaattgctatcgccatcacagctgaacttgttgagatccccgtgttactgcctatcagcattttactactttaaaaaaaaaaaaaaaagccaaaaaccaaatttgtatttaaggtatataaattttcccaaaactgataccctttgaaaaagtataaataaaatgagcaaaagttgaa

SEQ ID NO: 28 - DNA, 1776 bp, Homo sapiens
tttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcaaaaaaaaaaaaaaaaaa

(19) United States
(12) Patent Application Publication
(10) Pub. No.: US 2007/0237717 A1
(43) Pub. Date: Oct. 11, 2007

(54) Methods for Selection of Subjects for Multiple Sclerosis Therapy

(76) Inventors: Roland Martin, Bethesda, MD (US); Louis M. Staudt, Silver Spring, MD (US); Henry McFarland, Gaithersburg, MD (US); Claus-Steffen Sturzebecher, Berlin (DE)

Correspondence Address: KLARQUIST SPARKMAN, LLP, 121 S.W. SALMON STREET, SUITE #1600, PORTLAND, OR, 97204-2988, US

(21) Appl. No.: 11/578,207

(22) PCT Filed: Apr. 5, 2004
(86) PCT No.: PCT/US04/10584

§ 371 Date: Oct. 5, 2006

(51) Int. Cl. A61K 49/00; C12Q 1/68

(52) U.S. Cl. 424/9.1; 435/6

(57) ABSTRACT

A variety of therapies are used to treat autoimmune diseases such as multiple sclerosis. However, there is no single therapy that can be used to treat all subjects. Thus, a method is provided to determine if a subject with an autoimmune disease, such as multiple sclerosis, will respond to a therapeutic protocol. The method includes analyzing the expression of genes expressed by the immune system. Although the expression of a single gene can be assessed, such as interleukin-8, the methods include evaluating the expression profile of a subject using an array (such as a microarray) to determine if the subject is appropriately responding to the therapeutic protocol.

### FIELD

This disclosure relates to the treatment of multiple sclerosis, specifically to methods for identifying subjects with multiple sclerosis that are amenable to specific treatments, such interferon-beta (IFN-β).

### BACKGROUND

Multiple sclerosis (MS) is a chronic, neurological, autoimmune, demyelinating disease. MS can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent. In the United States there are about 250,000 to 400,000 persons with MS, and every week about 200 new cases are diagnosed. Worldwide, MS may affect 2.5 million individuals. Because it is not contagious, which would require U.S. physicians to report new cases, and because symptoms can be difficult to detect, the incidence of disease is only estimated and the actual number of persons with MS could be much higher.

The pathology of MS is characterized by an abnormal immune response directed against the central nervous system. In particular, T-lymphocytes are activated against the myelin sheath of the neurons of the central nervous system causing demyelination. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most subjects recover clinically from individual bouts of demyelination, producing the classic remitting and exacerbating course of the most common form of the disease known as relapsing-remitting multiple sclerosis.

MS develops in genetically predisposed individuals and is most likely triggered by environmental agents such as viruses (Martin et al., Ann. Rev. Immunol. 10:153-187, 1992). According to current hypotheses, activated autoreactive CD4+ T helper cells (Th1 cells) which preferentially secrete interferon-gamma (IFN-γ) and tumor necrosis factors alpha/beta (TNF-α/β), induce inflammation and demyelination in MS (Martin et al., supra). Proinflammatory cytokines (such as IFN-γ, TNF-α/β) and chemokines secreted by Th1 cells contribute to many aspects of lesion development including opening of the blood-brain-barrier, recruitment of other inflammatory cells, activation of resident glia (micro- and astroglia) and the effector phase of myelin damage via nitrogen and oxygen radicals secreted by activated macrophages (Wekerle et al., Trends Neuro Sci. 9:271-277, 1986; Martin et al., supra).

The status of MS patients can be evaluated by longitudinal, monthly follow-up of magnetic resonance imaging (MRI) activity in the brain of MS patients. MRI offers a unique set of outcome measures for phase I/II clinical trials in small cohorts of patients, and is thus well suited to establish data for proof of principle for novel therapeutic strategies (e.g., see Harris et al., Ann. Neurol. 29:548-555, 1991; MacFarland et al., Ann. Neurol. 32:758-766, 1992; Stone et al., Ann. Neurol. 37:611-619, 1995).

There are currently five approved treatments for relapsing-remitting MS, three types of IFN-β (the Interferon-B multiple sclerosis study group, Neurology 43:655-661, 1993; the IFNB Multiple Sclerosis Study Group and the University of British Columbia MS/MRI Analysis Group, Neurology 45:1277-1285, 1995; Jacobs et al., Ann. Neurol. 39:285-294, 1996), glatiramer-acetate, and an immunosuppressant, mitoxantrone (see Johnson K P, Group. tCMST, J. Neurol. 242:S38, 1995). Most patients are treated with IFN-β, but a large fraction of patients (approximately 50-60%) incompletely respond to treatment, completely fail treatment or develop non-responsiveness. Treatment failures have been linked to the development of neutralizing anti-IFN-β antibodies, although their role is also not completely understood at present (the IFNB Multiple Sclerosis Study Group and the University of British Columbia MS/MRI Analysis Group, Neurology 47:889-894, 1996). IFN-β therapy is expensive, and currently costs US$10,000 to US$12,000 per year. Similarly, other treatment protocols are costly. Thus, it would be extremely beneficial to develop a predictive test to determine if a patient will respond to a specific therapeutic protocol.

### SUMMARY

A variety of therapies are used to treat autoimmune diseases such as multiple sclerosis. However, there is no single therapy that can be used to treat all subjects. Thus, a method is provided to determine if a subject with an autoimmune disease, such as multiple sclerosis, will respond to a therapeutic protocol. The method includes analyzing the expression of genes expressed by the immune system. Although the expression of a single gene can be assessed, the methods include evaluating the expression profile of a subject using an array (such as a microarray) to determine if the subject is appropriately responding to the therapeutic protocol.

In one embodiment, a method is provided for determining if a subject with multiple sclerosis will respond to a therapeutic protocol. The method includes creating a cDNA probe from mRNA of lymphocytes isolated from the subject, hybridizing the probe to a microarray comprising gene sequences, determining the extent of hybridization of the probes to each gene on the microarray. A pattern of hybridization of the probes on the microarray indicates that the subject with multiple sclerosis will respond to the therapeutic protocol. In several examples, the therapeutic protocol includes treatment with interferon beta and/or with an antibody that binds the interleukin-2 receptor. In another example, the method includes determining the expression of a nucleic acid encoding IL-8.

In another embodiment, a method is provided for determining if a subject with multiple sclerosis will respond to a therapeutic protocol. The method includes hybridizing cDNA probes created from mRNAs of lymphocytes isolated from the subject to a microarray comprising nucleic acid sequences. The microarray comprises the nucleic acid sequences encoding IL-8, Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, TRAIL, zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R). The extent of hybridization of the probes to nucleic acid sequences on the microarray is determined, wherein the pattern of hybridization of the probes to the microarray indicates that the subject with multiple sclerosis will respond to the therapeutic protocol.

In a further embodiment, a method is disclosed for determining if a subject with multiple sclerosis will respond to treatment with interferon-beta. In one example, the method includes contacting a lymphocyte from the subject with IFN-β in vivo or in vitro and detecting a decrease in expression of interleukin-8 by a lymphocyte from the subject as compared to the expression of interleukin-8 by a lymphocyte from the subject not treated with interferon-beta. A decrease in interleukin-8 expression demonstrates that the subject will respond to treatment with interferon beta. In one example, the method includes isolating peripheral blood mononuclear cells (PBMC) the subject, and contacting the PBMC with IFN-β.

In yet a further embodiment, a method is provided for determining if a subject with multiple sclerosis will respond to treatment with interferon-beta. The method includes treating a lymphocyte from the subject with interferon-beta in vivo or in vitro, and detecting an increase in expression of at least one protein or at least one mRNA encoding the protein from a lymphocyte isolated from the subject as compared to the expression of the protein or the mRNA by a lymphocyte isolated from a subject not treated with interferon-beta, wherein the at least one protein comprises Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL. An increase in expression of the protein or mRNA following a treatment with interferon-beta demonstrates that the subject will respond to treatment with interferon beta.

The foregoing and other features and advantages will become more apparent from the following detailed description of several embodiments, which proceeds with reference to the accompanying figures.

### BRIEF DESCRIPTION OF THE FIGURES

FIGS. 1A-C are graphs of treatment response. FIG. 1A is a graph showing the treatment response in patients with high numbers of total Gd-enhancing lesions at baseline. FIG. 1B is a graph showing the treatment response in patients with low numbers of total Gd-enhancing lesions at baseline. FIG. 1C is a graph showing the treatment response in two patients not optimally responding to IFN-β therapy from initiation of therapy (INR). FIG. 1D is a graph showing the treatment response in two patients developing high titers of neutralizing antibodies. Data is shown for baseline and treatment on a 1-monthly and 2-monthly basis (except INR, with monthly MRIs displayed for treatment as well). Time points with high NAb titers are indicated by arrows labeled NAb. Circles with numbers indicate the time periods of sampling for cDNA array; samples from two to three time points were pooled in some cases. (Baseline sample for INR 2 was collected before the first MRI presented in the figure.)

FIGS. 2A-C are digital images showing that genes regulated ex vivo are not regulated during phases of non-response. Multifold of expression is represented by the intensity of color ranging between 2-2 for light gray to 22 for dark black. FIG. 2A is a digital image showing the results from eight in vitro experiments. FIG. 2B is a digital image showing ex vivo data from six responder patients (nine experiments) and two patients who later developed neutralizing antibodies (four experiments). FIG. 2C is a digital image of the ex vivo results during status of non-response. Two patients did not optimally respond from initiation of therapy (INR), two developed neutralizing antibodies (NAbNR) (total of four experiments).

FIG. 3 is a digital image showing the pattern of genes regulated by in vitro incubation with IFN-β for 24 hours. Multifold of expression versus untreated baseline is represented by the intensity of color ranging between 2-5 for light to 25 for dark. Asterisk indicates NAb patient samples. ‘+’ indicates the patient not optimally responding from beginning of therapy.

FIG. 4 is a graph of the longitudinal follow-up of IL-8 gene expression before and during IFN-β treatment. Treatment responders show downregulation of gene expression; in contrast the patients not fully responding to therapy demonstrate increased IL-8 expression (INR) or no change (NAbNR). Fold changes are shown on a log scale. B=baseline; T=treatment.

### SEQUENCE LISTING

The nucleic acids listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. A listing of the sequences provided in the sequence listing is presented in Table 3 (see the Examples section).

### DETAILED DESCRIPTION

### I. Abbreviations

AIM2: interferon inducible protein AIM2 (absent in melanoma);

AREB6: zinc finger homeodomain protein

Bag-1: Bcl-2 interacting anti-apoptotic protein

BNIP3: Bcl-2-interacting protein

BTG2: p53 dependent anti-proliferative gene

CDR: complementarity determining region

CBC: complete blood count

c-IAP2: inhibitor of apoptosis

CNS: central nervous system

DUSP5: dual specificity phosphatase induced by serum

EDSS: expanded disability status scale

EVI2B: ecotropic viral integration site 2 B protein

FLAME 1: FLICE-like inhibitory protein long

FR: framework region

GADD 45 beta: growth arrest and DNA damage inducible protein

GADD 153: growth arrest and DNA damage inducible gene

Gd: gadolinium

GLiPR: glioma pathogenesis-related protein

HEMAS: interferon stimulated gene 20 kDa

HIV: human immunodeficiency virus

HM74: G protein coupled receptor

hSEC10p: brain secretory protein

hSTYXb: tyrosine phosphatase-like protein

HV: hypervariable region

IFN: interferon

Ig: immunoglobulin

IκB-alpha: NF-κB inhibitory protein.

IEX-1L: immediate early gene (apoptosis inhibitor);

IL-2: interleukin 2

IL-2R: interleukin 2 receptor

INR: initial non-responders

JNK1: stress-activated protein kinase

kg: kilogram

KLH: keyhole limpet hemocyanin

LPS: lipopolysaccharide

MBP: myelin basic protein

MDS01: phorbolin I paralogue

MEF2A: MADS/MEF2 (myocyte enhancer-binding factor)

mg: milligram

MKP1: MAP kinase phosphatase 1

MLC: mini-lymphochip

mm: millimeter

MOG: myelin/oligodendrocyte glycoprotein

MRI: magnetic resonance imaging

MS: multiple sclerosis

NAbNR: neutralizing antibody non-responder

Nak1: nuclear receptor subfamily 4, group A (orphan steroid receptor);

NK: natural killer

NO—: nitric oxide

p19: cyclin-dependent kinase 4 inhibitor

PAC-1: protein tyrosine phosphatase

PBMC: peripheral blood mononuclear cells

PCR: polymerase chain reaction

PLP: myelin proteolipid protein

PCNA⁺: proliferating cell nuclear antigen positive

PNS: peripheral nervous system

RFU: relative fluorescent units

RT-PCR: reverse transcriptase polymerase chain reaction

Sgk: putative serine/threonine protein kinase

SRS: Scripps Neurological Rating Scale

TGF: transforming growth factor

TNF: tumor necrosis factor

TRADD: TNF receptor-1-associated protein

TSC-22R: transforming growth factor beta stimulated clone 22 related gene

TTP: tristetraproline

VRK2 kinase: vaccina virus kinase related kinase

VH: variable heavy

VL: variable light

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Antisense, Sense and Antigene: DNA has two strands, a 5′→3′ strand, referred to as the plus strand, and a 3′→5′ strand, referred to as the minus strand. Because RNA polymerase adds nucleic acids in a 5′→3′ direction, the minus strand of the DNA serves as the template for the RNA during transcription. Thus, the RNA formed will have a sequence complementary to the minus strand, and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a DNA target. An antisense RNA (asRNA) is a molecule of RNA complementary to a sense (encoding) nucleic acid molecule.

Array: An arrangement of molecules, particularly biological macromolecules (such as polypeptides or nucleic acids) in addressable locations on a substrate. The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized so as to require or benefit from microscopic examination, or other magnification, for its evaluation. Further miniaturization can be used to produce “nanoarrays.”

Within an array, each arrayed molecule is addressable, in that its location can be reliably and consistently determined within the at least two dimensions of the array surface. In ordered arrays, the location of each molecule sample can be assigned to the sample at the time when it is spotted or otherwise applied onto the array surface, and a key may be provided in order to correlate each location with the appropriate target. Often, ordered arrays are arranged in a symmetrical grid pattern, but samples could be arranged in other patterns (e.g., in radially distributed lines, spiral lines, or ordered clusters). Addressable arrays are computer readable, in that a computer can be programmed to correlate a particular address on the array with information (such as hybridization or binding data, including for instance signal intensity). In some examples of computer readable formats, the individual “spots” on the array surface will be arranged regularly in a pattern (e.g., a Cartesian grid pattern) that can be correlated to address information by a computer.

The sample application “spot” on an array may assume many different shapes. Thus, though the term “spot” is used, it refers generally to a localized deposit of nucleic acid, and is not limited to a round or substantially round region. For instance, substantially square regions of mixture application can be used with arrays encompassed herein, as can be regions that are substantially rectangular (such as a slot blot-type application), or triangular, oval or irregular. The shape of the array substrate itself is also immaterial, though it is usually substantially flat and may be rectangular or square in general shape.

Adverse effects: Any undesirable signs, including the clinical manifestations of abnormal laboratory results, or medical diagnoses noted by medical personnel, or symptoms reported by the subject that have worsened. Adverse events include, but are not limited to, life-threatening events, an event that prolongs hospitalization, or an event that results in medical or surgical intervention to prevent an undesirable outcome.

Antagonist of an IL-2 Receptor (IL-2R): An agent that specifically binds to the IL-2R, or a component thereof, and inhibits a biological function of the IL-2 receptor or the component. Exemplary functions that can be inhibited are the binding of IL-2 to the IL-2R, the intracellular transmission of a signal from binding of IL-2, and proliferation and/or activation of lymphocytes such as T cells in response to IL-2. In one embodiment, IL-2R antagonists of use in the methods disclosed herein inhibit at least one of these functions. Alternatively, IL-2R antagonists of use in the methods disclosed herein can inhibit more than one or all of these functions.

In one example, an IL-2 receptor antagonist is an antibody that specifically binds Tac (p55), such as Zenapax® (see below). Other anti-p55 agents include the chimeric antibody basiliximab (Simulect®), BT563 (see Baan et al., Transplant. Proc. 33:224-2246, 2001), and 7G8. Basiliximab has been reported to be beneficial in preventing allograft rejection (Kahan et al., Transplantation 67:276-84, 1999) and in treating psoriasis (Owen & Harrison, Clin. Exp. Dermatol. 25:195-7, 2000). An exemplary human anti-p55 antibody of use in the methods of the disclosure is HuMax-TAC, being developed by Genmab. In another example, an IL-2 receptor antagonist is an antibody that specifically binds the p75 or β subunit of the IL-2R.

Additional antibodies that specifically bind the IL-2 receptor are known in the art. For example, see U.S. Pat. No. 5,011,684; U.S. Pat. No. 5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559. The mik-β1 antibody is an antagonist that specifically binds the beta chain of human IL-2R.

In another example, an IL-2 receptor antagonist is a peptide antagonist that is not an antibody. Peptide antagonists of the IL-2 receptor, including antagonists of Tac (p55) and p75 (IL-2Rβ, CD122) are also known. For example, peptide antagonists for p55 and p75 are disclosed in U.S. Pat. No. 5,635,597. These peptides are also of use in the methods disclosed herein.

In a further example, an IL-2 receptor antagonist is a chemical compound or small molecule that specifically binds to the IL-2 receptor and inhibits a biological function of the receptor.

Antibody fragment (fragment with specific antigen binding): Various fragments of antibodies have been defined, including Fab, (Fab′)₂, Fv, and single-chain Fv (scFv). These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction or equivalently by genetic engineering; (4) F(Ab′)₂, a dimer of two Fab′ fragments held together by disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine in the art.

Autoimmune disorder: A disorder in which the immune system produces an immune response (e.g. a B cell or a T cell response) against an endogenous antigen, with consequent injury to tissues.

Beta interferon: Any beta interferon including interferon-beta 1a and interferon-beta 1b.

Interferon-beta 1a is a 166 amino acid glycoprotein with a predicted molecular weight of approximately 22,500 daltons. The interferon-beta 1a known as Avonex® is produced by recombinant DNA technology utilizing mammalian cells (Chinese Hamster Ovary cells) into which the human interferon-beta gene has been introduced. The amino acid sequence of Avonex® is identical to that of natural human interferon-beta. Interferon-induced gene products and markers including 2′,5′-oligoadenylate synthetase, β₂-microglobulin, and neopterin, have been measured in the serum and cellular fractions of blood collected from patients treated with Avonex®. Avonex® was approved in 1996 and is marketed by Biogen, Inc. Avonex® has been demonstrated to decrease the number of gadolinium (Gd)-enhanced lesions in subjects who were administered the drug for two years by up to 13%, and to improve approximately 22% of subjects' Expanded Disability Status Scale (EDSS) scores.

Another interferon-beta 1a was approved in 2002, marketed by Serono, Inc. This interferon-beta 1a, known as Rebif®, has recently been approved for treatment of relapsing-remitting MS. The primary difference between Avonex® and Rebif® is the approved method of administration—intramuscular injection for the former and subcutaneous injection for the latter. According to Samkoff, Hosp. Phys., p. 21-27, 2002, Rebif® can reduce relapse rates by 33% in subjects taking the drug.

Interferon-beta 1b is a highly purified protein that has 165 amino acids and an approximate molecular weight of 18,500 daltons. An interferon-beta 1b known as Betaseron® was approved as a treatment for MS in 1993 and is marketed by Berlex Laboratories, Inc. Betaseron® is manufactured by bacterial fermentation of a strain of Escherichia coli that bears a genetically engineered plasmid containing the gene for human interferon-beta. The native gene was obtained from human fibroblasts and altered to substitute serine for the cysteine residue found at position 17. According to the Physicians' Desk Reference (1996), Betaseron® has been demonstrated to reduce the exacerbation rate in subjects taking the drug by about 31%. The mechanisms by which interferon-beta 1b exerts its actions in multiple sclerosis are not clearly understood. However, it is known that the biologic response-modifying properties of interferon-beta 1b are mediated through its interactions with specific cell receptors. The binding of interferon-beta 1b to these receptors induces the expression of a number of interferon induced gene products (e.g., 2′,5′-oligoadenylate synthetase, protein kinase, and indolamine 2,3-dioxygenase) that are believed to be the mediators of the biological actions of interferon-beta 1b.

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecules. cDNA is usually synthesized in the laboratory by reverse transcription from messenger RNA extracted from cells or other samples.

Complementarity-determining region (CDR): The CDRs are three hypervariable regions within each of the variable light (VL) and variable heavy (VH) regions of an antibody molecule that form the antigen-binding surface that is complementary to the three-dimensional structure of the bound antigen. Proceeding from the N-terminus of a heavy or light chain, these complementarity-determining regions are denoted as “CDR1”, “CDR2,” and “CDR3,” respectively. CDRs are involved in antigen-antibody binding, and the CDR3 comprises a unique region specific for antigen-antibody binding. An antigen-binding site, therefore, may include six CDRs, comprising the CDR regions from each of a heavy and a light chain V region. Alteration of a single amino acid within a CDR region can destroy the affinity of an antibody for a specific antigen (see Abbas et al., Cellular and Molecular Immunology, 4th ed. 143-5, 2000). The locations of the CDRs have been precisely defined, e.g., by Kabat et al., Sequences of Proteins of Immunologic Interest, U.S. Department of Health and Human Services, 1983.

Complementarity and Percentage Complementarity (nucleic acid sequence): Molecules with complementary nucleic acids form a stable duplex or triplex when the strands bind, or hybridize, to each other by forming Watson-Crick, Hoogsteen or reverse Hoogsteen base pairs. Stable binding occurs when an oligonucleotide remains detectably bound to a target nucleic acid sequence under the required conditions.

Complementarity is the degree to which bases in one nucleic acid strand base pair with the bases in a second nucleic acid strand. Complementarity is conveniently described by the percentage, i.e., the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

“Sufficient complementarity” means that a sufficient number of base pairs exist between the oligonucleotide and the target sequence to achieve detectable binding, and disrupt or reduce expression of the gene product(s) encoded by that target sequence. When expressed or measured by percentage of base pairs formed, the percentage complementarity that fulfills this goal can range from as little as about 50% complementarity to full (100%) complementary. In some embodiments, sufficient complementarity is at least about 50%, about 75% complementarity, or at least about 90% or 95% complementarity. In particular embodiments, sufficient complementarity is 98% or 100% complementarity.

A thorough treatment of the qualitative and quantitative considerations involved in establishing binding conditions that allow one skilled in the art to design appropriate oligonucleotides for use under the desired conditions is provided by Beltz et al., Methods Enzymol 100:266-285, 1983; and by Sambrook et al. (ed.), Molecular Cloning: A Laboratory Manual, 2nd ed., vol. 1-3, Cold Spring Harbor Laboratory Press, Cold Spring Harbor, N.Y., 1989.

Epitope: The site on an antigen recognized by an antibody as determined by the specificity of the amino acid sequence. Two antibodies are said to bind to the same epitope if each competitively inhibits (blocks) binding of the other to the antigen as measured in a competitive binding assay (see, e.g., Junghans et al., Cancer Res. 50:1495-1502, 1990). Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

Fluorophore: A chemical compound which, when excited by exposure to a particular wavelength of light, emits light (i.e., fluoresces), for example at a different wavelength than that to which it was exposed. Fluorophores can be described in terms of their emission profile, or “color.” Green fluorophores, for example Cy3, FITC, and Oregon Green, are characterized by their emission at wavelengths generally in the range of 515-540λ Red fluorophores, for example Texas Red, Cy5 and tetramethylrhodamine, are characterized by their emission at wavelengths generally in the range of 590-690λ

Encompassed by the term “fluorophore” are luminescent molecules, which are chemical compounds which do not require exposure to a particular wavelength of light to fluoresce; luminescent compounds naturally fluoresce. Therefore, the use of luminescent signals eliminates the need for an external source of electromagnetic radiation, such as a laser. An example of a luminescent molecule includes, but is not limited to, aequorin (Tsien, Ann. Rev. Biochem. 67:509, 1998).

Examples of fluorophores are provided in U.S. Pat. No. 5,866,366. These include: 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenyl-azophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron®. Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other fluorophores include thiol-reactive europium chelates that emit at approximately 617 nm (Heyduk and Heyduk, Analyt. Biochem. 248:216-227, 1997).

Still other fluorophores include cyanine, merocyanine, styryl, and oxonyl compounds, such as those disclosed in U.S. Pat. No. 5,268,486; U.S. Pat. No. 5,486,616; U.S. Pat. No. 5,627,027; U.S. Pat. No. 5,569,587; and U.S. Pat. No. 5,569,766, and in published PCT Patent Application No. PCT/US98/00475, each of which is incorporated herein by reference. Specific examples of fluorophores disclosed in one or more of these patent documents include Cy3 and Cy5, for instance.

Other fluorophores include GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al., herein incorporated by reference) and derivatives thereof. Other fluorophores are known to those skilled in the art, for example those available from Molecular Probes (Eugene, Oreg.).

Framework region (FR): Relatively conserved sequences flanking the three highly divergent complementarity-determining regions (CDRs) within the variable regions of the heavy and light chains of an antibody. Hence, the variable region of an antibody heavy or light chain consists of an FR and three CDRs. Some FR residues may contact bound antigen; however, FRs are primarily responsible for folding the variable region into the antigen-binding site, particularly the FR residues directly adjacent to the CDRs. Without being bound by theory, the framework region of an antibody serves to position and align the CDRs. The sequences of the framework regions of different light or heavy chains are relatively conserved within a species. A “human” framework region is a framework region that is substantially identical (about 85% or more, usually 90-95% or more) to the framework region of a naturally occurring human immunoglobulin.

Immunoglobulin: A protein including one or more polypeptides substantially encoded by immunoglobulin genes. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acids in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17:105, 1987; Huston et al., Proc. Natl. Acad. Sci. U.S.A., 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986).

An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody (e.g., ATCC Accession No. CRL 9688 secretes an anti-Tac chimeric antibody), although other mammalian species can be used, or the variable region can be produced by molecular techniques. Methods of making chimeric antibodies are well known in the art, e.g., see U.S. Pat. No. 5,807,715, which is herein incorporated by reference.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr (see U.S. Pat. No. 5,585,089, which is incorporated herein by reference). Humanized immunoglobulins can be constructed by means of genetic engineering, e.g., see U.S. Pat. No. 5,225,539 and U.S. Pat. No. 5,585,089, which are herein incorporated by reference.

A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO 91/17271; McCafferty et al., PCT Publication No. WO 92/001047; and Winter, PCT Publication No. WO 92/20791, which are herein incorporated by reference), or selected from a human combinatorial monoclonal antibody library (see the Morphosys website). Human antibodies can also be prepared by using transgenic animals carrying a human immunoglobulin gene (e.g., see Lonberg et al., PCT Publication No. WO 93/12227; and Kucherlapati, PCT Publication No. WO 91/10741, which are herein incorporated by reference).

Interleukin 2 (IL-2): A protein of 133 amino acids (15.4 kDa) with a slightly basic pI that does not display sequence homology to any other factors. Murine and human IL-2 display a homology of approximately 65%. IL-2 is synthesized as a precursor protein of 153 amino acids with the first 20 amino terminal amino acids functioning as a hydrophobic secretory signal sequence. The protein contains a single disulfide bond (positions Cys58/105) essential for biological activity. The human IL-2 gene contains four exons and maps to human chromosome 4q26-28 (murine chromosome 3).

The biological activities of IL-2 are mediated by a membrane receptor that is expressed on activated, but not on resting, T cells and natural killer (NK) cells. Activated B cells and resting mononuclear leukocytes also rarely express this receptor.

IL-2 receptor: A cellular receptor that binds IL-2 and mediates its biological effects. Three different types of IL-2 receptors are distinguished that are expressed differentially and independently. The high affinity IL-2 receptor (K_{d}˜10 pM) constitutes approximately 10% of all IL-2 receptors expressed by cells. This receptor is a membrane receptor complex consisting of the two subunits: IL-2R-alpha (also known as T cell activation (TAC) antigen or p55) and IL-2R-beta (also known as p75 or CD122). An intermediate affinity IL-2 receptor (K_{d}=100 pM) consists of the p75 subunit and a gamma chain, while a low affinity receptor (K_{d}=10 nM) is formed by p55 alone.

p75 is 525 amino acids in length. It has an extracellular domain of 214 amino acids and a cytoplasmic domain of 286 amino acids. The p75 gene maps to human chromosome 22q11. 2-q12, contains 10 exons and has a length of approximately 24 kb. p55 is 251 amino acids in length with an extracellular domain of 219 amino acids and a very short cytoplasmic domain of 13 amino acids. The gene encoding p55 maps to human chromosome 10p14-p15.

p75 is expressed constitutively on resting T-lymphocytes, NK cells, and a number of other cell types while the expression of p55 is usually observed only after activation. Activated lymphocytes continuously secrete a 42 kDa fragment of p55 (TAC antigen). This fragment circulates in the serum and plasma, and functions as a soluble IL2 receptor (see Smith, Ann. Rev. Cell Biol. 5:397-425, 1989; Taniguchi and Minami, Cell 73:5-8, 1993).

p55 has a length of 251 amino acids with an extracellular domain of 219 amino acids and a very short cytoplasmic domain of 13 amino acids. The p55 gene maps to human chromosome 10p14-p15. The expression of p55 is regulated by a nuclear protein called RPT-1.

A third 64 kDa subunit of the IL2 receptor, designated gamma, has been described. This subunit is required for the generation of high and intermediate affinity IL-2 receptors but does not bind IL-2 by itself. The gene encoding the gamma subunit of the IL2 receptor maps to human chromosome Xq13, spans approximately 4.2 kb and contains eight exons.

IL-8: IL-8 is a non-glycosylated protein of 8 kDa (72 amino acids). It is produced by processing of a precursor protein of 99 amino acids. IL-8 is produced by stimulated monocytes but not by tissue macrophages and T-lymphocytes. IL-8 is produced also by macrophages, fibroblasts, endothelial cells, keratinocytes, melanocytes, hepatocytes, chondrocytes, and a number of tumor cell lines.

The human IL8 gene has a length of 5.1 kb and contains four exons. It maps to human chromosome 4q12-q21. The mRNA consists of a 101 base 5′ untranslated region, an open reading frame of 297 bases, and a long 3′ untranslated region of 1.2 kb.

The activities of IL-8 are not species-specific. Human IL-8 is also active in rodent and rabbit cells. The biological activities of IL-8 resemble those of a related protein, neutrophil-activating peptide-2. IL-8 has the ability to specifically activate neutrophil granulocytes. IL-8 antagonizes IgE production by human B cells. IL-8 is chemotactic for all known types of migratory immune cells. IL-8 can be detected in assays measuring the migration of buff coat leukocytes from agarose blocks. Sensitive immunoassays are also available.

In vitro amplification: Techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Magnetic Resonance Imaging: A noninvasive diagnostic technique that produces computerized images of internal body tissues and is based on nuclear magnetic resonance of atoms within the body induced by the application of radio waves.

Brain MRI is an important tool for understanding the dynamic pathology of multiple sclerosis. T₂-weighted brain MRI defines lesions with high sensitivity in multiple sclerosis and is used as a measure of disease burden. However, such high sensitivity occurs at the expense of specificity, as T₂ signal changes can reflect areas of edema, demyelination, gliosis and axonal loss. Areas of gadolinium (Gd) enhancement demonstrated on T₁-weighted brain MRI are believed to reflect underlying blood-brain barrier disruption from active perivascular inflammation. Such areas of enhancement are transient, typically lasting <1 month. Gadolinium-enhanced T₁-weighted brain MRI are therefore used to assess disease activity. Most T₂-weighted (T₂) lesions in the central white matter of subjects with multiple sclerosis begin with a variable period of T₁-weighted (T₁) gadolinium (Gd) enhancement and that T₁ Gd-enhancing and T₂ lesions represent stages of a single pathological process. The brain MRI techniques for assessing T₁ and T₂ Gd-enhancing lesions are standard (e.g., see Lee et al., Brain 122 (Pt 7):1211-2, 1999).

Monoclonal antibody: An antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells.

Multiple sclerosis: An autoimmune disease classically described as a central nervous system white matter disorder disseminated in time and space that presents as relapsing-remitting illness in 80-85% of patients. The diagnosis is made from the clinical examination and history or also from typical MRI findings in the brain and spinal cord. Somatosensory and visual evoked potential and analysis of cerebrospinal fluid to detect the amount of immunoglobulins and oligoclonal bands are considered supportive findings. MRI is a particularly sensitive diagnostic tool. MRI abnormalities indicating the presence or progression of MS include hyperintense white matter signals on T₂-weighted and fluid attenuated inversion recovery images, gadolinium enhancement of active lesions, hypointensive “black holes” (representing gliosis and axonal pathology), and brain atrophy on T₁-weighted studies. Serial MRI studies can be used to indicate disease progression.

Relapsing-remitting (RR) multiple sclerosis is a clinical course of MS that is characterized by clearly defined, acute attacks with full or partial recovery and no disease progression between attacks.

Secondary-progressive multiple sclerosis is a clinical course of MS that initially is relapsing-remitting, and then becomes progressive at a variable rate, possibly with an occasional relapse and minor remission.

Primary progressive multiple sclerosis presents initially in the progressive form.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Inosine is also a base that can be integrated into DNA or RNA in a nucleotide (dITP or ITP, respectively).

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Open reading frame: A series of nucleotide triplets (codons) coding for amino acids without any internal termination codons. These sequences are usually translatable into a peptide.

Ortholog: Two nucleic acid or amino acid sequences are orthologs of each other if they share a common ancestral sequence and diverged when a species carrying that ancestral sequence split into two species. Orthologous sequences are also homologous sequences.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Polymerization: Synthesis of a nucleic acid chain (oligonucleotide or polynucleotide) by adding nucleotides to the hydroxyl group at the 3′-end of a pre-existing RNA or DNA primer using a pre-existing DNA strand as the template. Polymerization usually is mediated by an enzyme such as a DNA or RNA polymerase. Specific examples of polymerases include the large proteolytic fragment of the DNA polymerase I of the bacterium E. coli (usually referred to as Kleenex polymerase), E. coli DNA polymerase I, and bacteriophage T7 DNA polymerase. Polymerization of a DNA strand complementary to an RNA template (e.g., a cDNA complementary to an mRNA) can be carried out using reverse transcriptase (in a reverse transcription reaction).

For in vitro polymerization reactions, it is necessary to provide to the assay mixture an amount of required cofactors such as M⁺⁺, and dATP, dCTP, dGTP, dTTP, ATP, CTP, GTP, UTP, or other nucleoside triphosphates, in sufficient quantity to support the degree of polymerization desired. The amounts of deoxyribonucleotide triphosphates substrates required for polymerizing reactions are well known to those of ordinary skill in the art. Nucleoside triphosphate analogs or modified nucleoside triphosphates can be substituted or added to those specified above.

Primer: Primers are relatively short nucleic acid molecules, usually DNA oligonucleotides, six nucleotides or more in length. Primers can be annealed to a complementary target DNA strand (“priming”) by nucleic acid hybridization to form a hybrid between the primer and the target DNA strand, and then the primer extended along the target DNA strand by a nucleic acid polymerase enzyme. Pairs of primers can be used for amplification of a nucleic acid sequence, e.g., by nucleic-acid amplification methods known in to those of ordinary skill in the art.

A primer is usually single stranded, which may increase the efficiency of its annealing to a template and subsequent polymerization. However, primers also may be double stranded. A double stranded primer can be treated to separate the two strands, for instance before being used to prime a polymerization reaction (see for example, Nucleic Acid Hybridization. A Practical Approach. Hames and Higgins, eds., IRL Press, Washington, 1985). By way of example, a double stranded primer can be heated to about 90°-100° C. for about 1 to 10 minutes.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “fragment” refers to a portion of a polypeptide that is at least 8, 10, 15, 20 or 25 amino acids in length. The term “functional fragments of a polypeptide” refers to all fragments of a polypeptide that retain an activity of the polypeptide (e.g., the binding of an antigen). Biologically functional fragments, for example, can vary in size from a polypeptide fragment as small as an epitope capable of binding an antibody molecule to a large polypeptide capable of participating in the characteristic induction or programming of phenotypic changes within a cell. The term “soluble” refers to a form of a polypeptide that is not inserted into a cell membrane.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful in the methods disclosed herein are conventional. Remington's Pharmaceutical Sciences, by E. W. Martin, Mack Publishing Co., Easton, Pa., 15th Edition (1975), describes compositions and formulations suitable for pharmaceutical delivery of the IL-2 receptor antagonists herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

Probes and primers: Nucleic acid probes and primers can be readily prepared based on the nucleic acid molecules provided in this disclosure as indicators of disease or disease progression. It is also appropriate to generate probes and primers based on fragments or portions of these nucleic acid molecules. Also appropriate are probes and primers specific for the reverse complement of these sequences, as well as probes and primers to 5′ or 3′ regions.

A probe comprises an isolated nucleic acid attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., in Sambrook et al. (in Molecular Cloning: A Laboratory Manual, CSHL, New York, 1989) and Ausubel et al. (in Current Protocols in Molecular Biology, John Wiley & Sons, New York, 1998).

Primers are short nucleic acid molecules, for instance DNA oligonucleotides 10 nucleotides or more in length. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using nucleic acid probes and primers are described, for example, in Sambrook et al. (in Molecular Cloning: A Laboratory Manual, CSHL, New York, 1989), Ausubel et al. (ed.) (in Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, for example, a primer comprising 30 consecutive nucleotides of a protein encoding nucleotide will anneal to a target sequence, such as another homolog of the designated protein, with a higher specificity than a corresponding primer of only 15 nucleotides. Thus, in order to obtain greater specificity, probes and primers can be selected that comprise at least 20, 25, 30, 35, 40, 45, 50 or more consecutive nucleotides of a protein-encoding nucleotide sequence.

The disclosure thus includes isolated nucleic acid molecules that comprise specified lengths of the disclosed nucleotide sequences. Such molecules may comprise at least 10, 15, 20, 23, 25, 30, 35, 40, 45 or 50 consecutive nucleotides of these sequences or more, and may be obtained from any region of the disclosed sequences (e.g., a nucleic acid may be apportioned into halves or quarters based on sequence length, and isolated nucleic acid molecules may be derived from the first or second halves of the molecules, or any of the four quarters, etc.). A cDNA also can be divided into smaller regions, e.g. about eighths, sixteenths, twentieths, fiftieths and so forth, with similar effect.

Another mode of division is to select the 5′ (upstream) and/or 3′ (downstream) region associated with a gene.

Nucleic acid molecules may be selected that comprise at least 10, 15, 20, 25, 30, 35, 40, 50 or 100 or more consecutive nucleotides of any of these or other portions of a nucleic acid molecule, such as those disclosed herein, and associated flanking regions. Thus, representative nucleic acid molecules might comprise at least 10 consecutive nucleotides of a human coding sequence the expression of which is influenced by treatment with interferon-beta, such as the proteins identified in Table 1.

Purified: The term purified does not require absolute purity or isolation; rather, it is intended as a relative term. Thus, for example, a purified or isolated protein preparation is one in which protein is more enriched than the protein is in its generative environment, for instance within a cell or in a biochemical reaction chamber. Preferably, a preparation of protein is purified such that the protein represents at least 50% of the total protein content of the preparation. For pharmaceuticals, “substantial” purity of 90%, 95%, 98% or even 99% or higher of the active agent can be utilized.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

Representational difference analysis: A PCR-based subtractive hybridization technique used to identify differences in the mRNA transcripts present in closely related cell lines.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of the IL-2R antibodies or antigen binding fragments, and the corresponding cDNA sequence, will possess a relatively high degree of sequence identity when aligned using standard methods. This homology will be more significant when the orthologous proteins or cDNAs are derived from species that are more closely related, compared to species more distantly related (e.g., human and murine sequences).

Methods of alignment of sequences for comparison are well known in the art. Various programs and alignment algorithms are described in Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244 9, 1988); Higgins and Sharp, CABIOS 5:151-153, 1989; Corpet et al., Nuc. Acids Res. 16:10881-90, 1988; Huang et al., Computer Appls. in the Biosciences 8:155-65, 1992; and Pearson et al., Meth. Mol. Bio. 24:307-31, 1994. Altschul et al., J. Mol. Biol. 215:403-410, 1990, presents a detailed consideration of sequence alignment methods and homology calculations.

Specific binding agent: An agent that binds substantially only to a defined target. As used herein, the term includes antibodies and other agents that bind substantially only to a specified protein or a component thereof (e.g., p55, p75).

Antibodies may be produced using standard procedures described in a number of texts, including Harlow and Lane (Using Antibodies, A Laboratory Manual, CSHL, New York, 1999, ISBN 0-87969-544-7). In addition, certain techniques may enhance the production of neutralizing antibodies (U.S. Pat. No. 5,843,454; U.S. Pat. No. 5,695,927; U.S. Pat. No. 5,643,756; and U.S. Pat. No. 5,013,548). The determination that a particular agent binds substantially only to a specific component may readily be made by using or adapting routine procedures. One suitable in vitro assay makes use of the Western blotting procedure (described in many standard texts, including Harlow and Lane, Antibodies: A Laboratory Manual, Cold Spring Harbor Press, 1988). Western blotting may be used to determine that a given protein binding agent, such as a monoclonal antibody, binds substantially only to the protein of interest.

Shorter fragments of antibodies can also serve as specific binding agents. For instance, Fabs, Fvs, and single-chain Fvs (SCFvs) that bind to an IL-2 receptor would be IL-2 receptor-specific binding agents.

Serial analysis of gene expression: The use of short diagnostic sequence tags to allow the quantitative and simultaneous analysis of a large number of transcripts in tissue, as described in Velculescu et al. (Science 270:484-487, 1995).

Subject: A human or non-human animal. In one embodiment, the subject has multiple sclerosis.

A subject who has multiple sclerosis who has failed a therapeutic protocol (such as administration of interferon-beta) is a subject who does not respond or fails to respond adequately to the therapy, such that their condition has not improved sufficiently, not changed, or deteriorated in response to treatment with a therapeutically effective amount of the drug. A subject who has failed a therapeutic protocol can require escalating doses of the drug to achieve a desired effect.

In one example, the failure of a subject with MS to respond to a therapeutic agent, such as interferon-beta, can be measured as a recurrence of Gd-contrasting MRI lesions to at least half of the mean of the baseline monthly contrasting lesions over six months. In other examples, a subject with MS that fails to respond to a therapeutic agent, such as interferon-beta treatment, is identified by the subject experiencing one or more exacerbations in an 18 month period of interferon-beta therapy, exhibiting an increase of 1 point or more on the EDSS over 18 months of treatment, or having persistence or reoccurrence of contrast enhancing lesions on brain MRI scans to at least one-half the mean of a baseline of monthly contrast enhancing lesions established over a 6-month baseline period measured prior to the beginning of the interferon-beta therapy.

Without being bound by theory, a subject can fail to respond to IFN treatment due to the development of neutralizing antibodies, although a failure to respond to IFN treatment can also be detected in the absence of neutralizing antibodies (primary failure). In one example, a subject who fails treatment with interferon-beta is a subject who develops neutralizing antibodies that specifically bind interferon-beta, such that escalating doses are required to see an effect, or to alter a sign or symptom of MS.

Symptom and sign: Any subjective evidence of disease or of a subject's condition, i.e., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to, any measurable parameters such as tests for immunological status or the presence of lesions in a subject with multiple sclerosis.

Therapeutically Effective Amount: A dose sufficient to prevent advancement, or to cause regression of the disease, or which is capable of reducing symptoms caused by the disease, such as multiple sclerosis.

Zenapax® (daclizumab): A particular recombinant, humanized monoclonal antibody of the human IgG1 isotype that specifically binds Tac (p55). The recombinant genes encoding Zenapax® are a composite of human (about 90%) and murine (about 10%) antibody sequences. The donor murine anti-Tac antibody is an IgG2a monoclonal antibody that specifically binds the IL-2R Tac protein and inhibits IL-2-mediated biologic responses of lymphoid cells. The murine anti-Tac antibody was “humanized” by combining the complementarity-determining regions and other selected residues of the murine anti-TAC antibody with the framework and constant regions of the human IgG1 antibody. The humanized anti-Tac antibody daclizumab is described and its sequence is set forth in U.S. Pat. No. 5,530,101, see SEQ ID NO: 5 and SEQ ID NO: 7 for the heavy and light chain variable regions respectively. U.S. Pat. No. 5,530,101 and Queen et al., Proc. Natl. Acad. Sci. 86:1029-1033, 1989, are both incorporated by reference herein in their entirety. Daclizumab inhibits IL-2-dependent antigen-induced T cell proliferation and the mixed lymphocyte response (MLR) (Junghans et al., Cancer Research 50:1495-1502, 1990), as can other antibodies of use in the methods disclosed herein.

Zenapax® has been approved by the U.S. Food and Drug Administration (FDA) for the prophylaxis of acute organ rejection in subjects receiving renal transplants, as part of an immunosuppressive regimen that includes cyclosporine and corticosteroids. Zenapax® has been shown to be well tolerated and effective in reducing T cell proliferation in patients with human T cell lymphotrophic virus type 1 associated myelopathy/topical spastic paraparesis (HAM/TSP, see Lehky et al., Ann. Neuro. 44:942-947, 1998). The use of Zenapax® to treat posterior uveitis has also been described (see Nussenblatt et al., Proc. Natl. Acad. Sci. 96:7462-7466, 1999).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III Description of Several Specific Embodiments

Provided herein are methods for determining if a subject with an autoimmune disorder, such as multiple sclerosis, will respond to a therapeutic protocol. Therapeutic protocols include, but are not limited to, therapies that involve treatment with interferon-beta or with an antibody that specifically binds the interleukin-2 receptor. Therapeutic protocols include those well known in the art and new experimental protocols.

The methods include detecting a change in expression of a nucleic acid expressed by the cells of the subject. The cells can be cells of the immune system. These nucleic acids include the nucleic acids encoding the proteins listed in Table 1, the nucleic acids encoding proteins in the general classes listed in Table 2, or the specific proteins listed in Table 2. Accession and sequence information for these sequences is provided in Table 3. The nucleic acid molecules include genes, cDNAs or other polynucleotide molecules encoding one of the listed proteins, or a fragment thereof.

In certain embodiments, abnormalities are detected in the expression of more than one nucleic acid, for instance in at least 2, at least 5, 10, 15, 25, 50, or 100 or more nucleic acid molecules listed in Tables 1 or 2, or elsewhere herein. In one embodiment, the nucleic acids are nucleic acids expressed by cells in the immune system and/or the nervous system. In certain specific embodiments, no more than the molecules listed in Tables-1-3, or corresponding to (represented by) those listed in Tables 1-3, are included in such analysis. For instance, certain of the described methods employ detecting no more than 5, no more than 10, no more than 20, no more than 50, or no more than 100 of such molecules.

In one embodiment, the methods disclosed herein include the use of an ordered array of nucleic acids representing thousands of genes on a solid support. mRNA from the cells of interest are used to create a labeled, first strand cDNA probe that is then hybridized to the microarray. In one embodiment, two mRNA samples are directly compared to the same microarray by incorporating different labels into the cDNA probes derived from the samples. The extent of hybridization of the probes to each nucleic acid sequence on the microarray is then quantitated and the ratio of the pixel intensities for each label is used as a measure of the relative mRNA expression in the two samples. In one embodiment, the array is an array of nucleic acids expressed by the nervous system and/or the immune system. In one example, a lymphochip is utilized, which includes nucleic acid sequences derived from high-throughput sequencing of cDNA clones from libraries of human immune cells. The array can incorporate, for example, thousands of clones from a library prepared from the immune system, such as from the germinal center B cells, lymphoma and leukemia subtypes. The array can also include genes of known structure and function based on their established role in immune cell differentiation, response, and disorders. These types of arrays are well known in the art (see, for example, Staudt, Trends Immunol. 22: 35-40, 2001; Staudt and Brown, Ann. Rev. Immunol. 18:829-859, 2000; Alizadeh et al., Nature 403:503-511, 2000; Alizadeh et al., Cold Spring Harbor Symp. Quant. Biol. 64:71-78, 1999; U.S. Patent Application No. 20030203416A1, all of which are incorporated herein by reference).

The array can be a high density array, such that the array includes greater than about 100, greater than about 1000, greater than about 16,000 and most greater than about 65,000 or 250,000 or even greater than about 1,000,000 different oligonucleotide probes. The oligonucleotide probes generally range from about 5 to about 50 nucleotides, such as about 10 to about 40 nucleotides in length or from about 15 to about 40 nucleotides in length.

The location and sequence of each different oligonucleotide probe sequence in the array is known. Moreover, in a high density array, the large number of different probes occupies a relatively small area so that there is a probe density of greater than about 60 different oligonucleotide probes per cm², such as greater than about 100, greater than about 600, greater than about 1000, greater than about 5,000, greater than about 10,000, greater than about 40,000, greater than about 100,000, or greater than about 400,000 different oligonucleotide probes per cm². The small surface area of the array (such as less than about 10 cm², less than about 5 cm², less than about 2 cm²) permits extremely uniform hybridization conditions (temperature regulation, salt content, etc.) while the extremely large number of probes allows parallel processing of hybridizations.

Generally, the methods of monitoring gene expression using array technology involve (1) providing a pool of target nucleic acids comprising RNA transcript(s) of one or more target gene(s), or nucleic acids derived from the RNA transcript(s); (2) hybridizing the nucleic acid sample to an array of probes (including control probes), that can be a high density array; and (3) detecting the hybridized nucleic acids and calculating a relative expression (transcription) level.

In order to measure the transcription level of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template, such as a cDNA (“first strand” transcribed from the mRNA). Thus, a cDNA reverse transcribed from an mRNA, an RNA transcribed from that cDNA, a DNA amplified from the cDNA, an RNA transcribed from the amplified DNA, etc., are all derived from the mRNA transcript. Detection of such products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, and the like.

Generally, the transcription level (and thereby expression) of one or more genes in a sample is quantified, so that the nucleic acid sample is one in which the concentration of the mRNA transcript(s) of the gene or genes, or the concentration of the nucleic acids derived from the mRNA transcript(s), is proportional to the transcription level (and therefore expression level) of that gene. The hybridization signal intensity should also be proportional to the amount of hybridized nucleic acid. Generally, the proportionality is relatively strict (for example, a doubling in transcription rate results in approximately a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity can be sufficient. Where more precise quantification is required, controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, controls or calibrations may not be required.

In one embodiment, a nucleic acid sample is utilized, such as the total mRNA isolated from a biological sample. The biological sample can be from any biological tissue or fluid from the subject of interest, such as a subject with an autoimmune disorder (for example, multiple sclerosis). Such samples include, but are not limited to, blood, blood cells (e.g., white cells), tissue biopsies, or fine needle biopsy samples. However, the sample could also be urine, peritoneal fluid, and pleural fluid, cerebral spinal fluid, tissue sample, single cells, or cells separated from a sample.

Nucleic acids (such as mRNA) may be isolated from the sample according to any of a number of methods well known to those of skill in the art. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993). In one example, the total nucleic acid is isolated from a given sample using, for example, an acid guanidinium-phenol-chloroform extraction method, and polyA+ mRNA is isolated by oligo dT column chromatography or by using (dT)n magnetic beads (see, e.g., Sambrook et al, Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, N.Y. (1987)).

The nucleic acid sample can be amplified prior to hybridization. If a quantitative result is desired, a method is utilized that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. The array can then include probes specific to the internal standard for quantification of the amplified nucleic acid.

Suitable amplification methods include, but are not limited to, polymerase chain reaction (PCR) (see Innis et al., PCR Protocols, A guide to Methods and Application, Academic Press, Inc. San Diego, 1990), ligase chain reaction (LCR) (see Wu and Wallace, Genomics 4:560, 1989; Landegren et al., Science 241:1077, 1988; and Barringer, et al., Gene 89:117, 1990), transcription amplification (Kwoh et al., Proc. Natl. Acad. Sci. U.S.A. 86:1173, 1989), and self-sustained sequence replication (Guatelli et al., Proc. Nat. Acad. Sci. U.S.A. 87:1874, 1990). In one embodiment, the sample mRNA is reverse transcribed with a reverse transcriptase and a primer consisting of oligo dT and a sequence encoding the phage T7 promoter to provide single stranded DNA template (termed “first strand”). The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA.

Methods of in vitro polymerization are well known to those of skill in the art (see, e.g., Sambrook, supra; Van Gelder et al., Proc. Natl. Acad. Sci. U.S.A. 87:1663-1667, 1990). The direct transcription method provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lamda vectors designed for Cre-loxP plasmid subcloning (see e.g., Palazzolo et al., Gene 88: 25-36, 1990).

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches.

One of skill in the art will appreciate that hybridization conditions can be designed to provide different degrees of stringency. In a one embodiment, hybridization is performed at low stringency in this case in 6×SSPE-T at 37° C. (0.005% Triton X-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. These steps have been standardized for commercially available array systems.

Methods for evaluating the hybridization results vary with the nature of the specific probe nucleic acids used as well as the controls provided. In one embodiment, simple quantification of the fluorescence intensity for each probe is determined. This is accomplished simply by measuring probe signal strength at each location (representing a different probe) on the array (for example, where the label is a fluorescent label, detection of the amount of florescence (intensity) produced by a fixed excitation illumination at each location on the array). Comparison of the absolute intensities of an array hybridized to nucleic acids from a “test” sample (such as from a patient treated with a therapeutic protocol) with intensities produced by a “control” sample (such as from the same patient prior to treatment with the therapeutic protocol) provides a measure of the relative expression of the nucleic acids that hybridize to each of the probes.

Changes in expression detected by these methods for instance can be different for different therapies, and may include increases or decreases in the level (amount) or functional activity of such nucleic acids, their expression, or in their localization or stability. An increase or a decrease can be, for example, about a 1-fold, 2-fold, 3-fold, 4-fold, 5-fold, change (increase or decrease) in the expression of a particular nucleic acid. In one non-limiting example, the molecule is expressed by the immune system and/or the nervous system. As used herein, the term “molecules expressed by the immune system” includes nucleic acid molecules (such as DNA or RNA or cDNA) and proteins expressed by any cell of the immune system, though in specific embodiments the term may be specific for any one of these types of molecules. The term is not limited to those molecules listed in Tables 1-3 (and molecules that correspond to those listed, or molecules that fall into the listed classes), but also includes other nucleic acids and/or proteins that are influenced (e.g., as to level, activity, localization) during autoimmune disease progression, including all of such molecules listed herein. It should be noted that these molecules can be expressed by cells other than the cells of the immune system, as long as they are also expressed by a cell in the immune system. Similarly, a “molecule expressed by the nervous system is expressed by cells in the nervous system, but can also be expressed by cells outside of the nervous system. One example of such a molecule expressed by the nervous system is tyrosine hydroxylase, which is expressed by some neurons by is also expressed by some pancreatic endocrine cells.

Certain of the encompassed methods involve measuring an amount of the molecule in a sample (such as a serum, blood or tissue sample) derived or taken from the subject, in which a difference (for instance, an increase or a decrease) in the level of the molecule relative to that present in a sample derived or taken from the subject at an earlier time, is diagnostic or prognostic for the usefulness of the specific therapeutic protocol. Certain of the encompassed methods involve measuring an amount of a molecule in a sample derived or taken from the subject, compared to the level of the molecule relative to that present in a control sample, such as a subject that correctly responds, or does not respond, to the therapeutic protocol of interest. Although this can be accomplished using nucleic acid arrays, it does not require the use of such a nucleic acid array.

Alterations, including increases or decreases in the expression of nucleic acid molecules can be detected using, for instance, in vitro nucleic acid amplification and/or nucleic acid hybridization. The results of such detection methods can be quantified, for instance by determining the amount of hybridization or the amount of amplification.

Alterations in expressed molecules can be detected using, for instance, a specific binding agent, such as an antibody, which in some instances will be detectably labeled. In certain embodiments, therefore, detecting an abnormality includes contacting a sample from the subject with a protein-specific binding agent, such as an antibody; and detecting whether the binding agent is bound by the sample. The level of the protein present in the sample is measured. A difference in the level of protein in the sample indicates if the therapeutic protocol is efficacious, or indicates if the therapeutic protocol is likely to reduce a sign or a symptom of the disorder. The level of the protein can be measured, relative to the level of the protein found an analogous sample from a subject not treated with the therapeutic protocol, a standard protein level in analogous samples, or a protein level from the same subject prior to treatment.

Specific embodiments of methods for detecting an alteration in the expression of an expressed nucleic acid use the arrays disclosed herein. Such arrays are nucleotide (e.g., polynucleotide, see above) or protein (e.g., peptide, polypeptide, or antibody) arrays. The array can be a high-density array. In such methods, an array may be contacted with polynucleotides or polypeptides (respectively) from (or derived from) a sample from a subject. The amount and/or position of binding of the subject's polynucleotides or polypeptides then can be determined, for instance, to produce a gene expression profile for that subject. Such gene expression profile can be compared to another gene expression profile, for instance a control gene expression profile from a subject not treated with a therapeutic protocol, or a subject who responds well to the therapeutic protocol. Optionally, the subject's gene expression profile (also known as a gene expression fingerprint) can be correlated with one or more appropriate treatments. Similarly, protein arrays can give rise to protein expression profiles. Both protein and gene expression profiles can more generally be referred to as expression profiles.

Other embodiments are methods that involve providing nucleic acids from a sample isolated from the subject; amplifying the nucleic acids to form nucleic acid amplification products; contacting the nucleic acid amplification products with an oligonucleotide probe that will hybridize under stringent conditions with a nucleic acid encoding a protein expressed by the immune system; detecting the nucleic acid amplification products which hybridize with the probe; and quantifying the amount of the nucleic acid amplification products that hybridize with the probe. The sequence of such oligonucleotide probes may be selected to bind specifically to a nucleic acid molecule listed in Tables 1-3. The primers may be selected to amplify a nucleic acid molecule listed in Tables 1-3. In specific examples of such methods, the primers are selected to amplify at least one nucleic acid product encoding IL-8, Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, TRAIL, zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κ B inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R).

Disclosed are methods of screening for a compound useful in treating, reducing, or preventing multiple sclerosis or development or progression of this disease. Such methods involve determining if application of a test compound alters a gene expression profile so that the profile more closely resembles the profile of a subject that does not have multiple sclerosis, or a subject that is responding well to therapy, and selecting a compound that so alters the gene expression profile. In specific examples of such methods, the test compound is applied to test lymphocytes, or is administered to the subject. In one embodiment the expression of at least one nucleic acid product encoding IL-8, Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, TRAIL, zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R) is assessed. In other embodiments, the expression of two or more of these nucleic acid products are assessed.

The disclosure is illustrated by the following non-limiting Examples.

### EXAMPLES

Multiple sclerosis is considered a T cell-mediated autoimmune disease that is characterized by CNS inflammation, demyelination and various degrees of axonal damage (McFarlin et al., New Engl J Med 307:1246-51, 1982; Trapp et al., New Engl J Med 338:278-85, 1998). Similar to diabetes and rheumatoid arthritis, genetic studies have demonstrated that major histocompatibility complex [MHC; human leucocyte antigen (HLA) in humans] class II alleles, as well as a considerable number of other as yet unidentified susceptibility genes, contribute to the quantitative genetic trait that predisposes to disease (Becker et al., Proc Natl Acad Sci U.S.A. 95:9979-84, 1998). Individual patients rarely carry all susceptibility genes. The genetic heterogeneity and possibly different environmental triggers, e.g. viral infections (Santoro et al., Cell 99:817-27, 1999), lead in turn to variability in disease patterns defined either clinically or pathologically (Lucchinetti et al., Ann Neurol 47:707-17, 2000). The same heterogeneity probably also contributes to differences in response to treatment.

There are currently three categories of approved therapies for the long-term therapy of relapsing-remitting (RR) multiple sclerosis, three different preparations of interferon (IFN)-β [IFN-β-1a (Avonex™), IFN-β-1b. (Betaseron™) and IFN-β-1a (Rebif™)], glatiramer-acetate (Copaxone™) and mitoxantrone (Novantrone™) [IFNB Multiple Sclerosis Study Group and The University of British Columbia MS/MRI Analysis Group, Neurology 45:1277-85, 1995; Johnson et al., Neurology 45:1268-76, 1995; Jacobs et al., Ann Neurol 39:285-94, 1996; PRISMS (Prevention of Relapses and Disability by Interferon beta-1a Subcutaneously in Multiple Sclerosis) Study Group, Lancet 352:1498-504, 1998; Bielekova and Martin, Curr Treat Options Neurol 1:201-20, 1999). Type I IFNs, including IFN-β, are not only part of the innate immune system and exert antiviral activities, but also cause complex immunomodulatory effects (Wandinger et al., Ann Neurol 50:349-57, 2001).

Recent advances in the development of genomics and proteomics offer opportunities to replace hypothesis-based biomarker studies by large-scale analyses that aim at capturing complex gene and protein expression responses (Alizadeh et al., Nature 403:503-11, 2000; Staudt and Brown, Annu Rev Immunol 18:829-59, 2000; Martin et al., Nat Immunol 2:785-8, 2001). The gene expression profiles in response to IFN-β therapy in multiple sclerosis patients were evaluated, and this data is presented herein. The in vitro pretreatment gene expression profile to IFN-β was used as a parameter for the baseline responsiveness. This was compared with the biological response in vivo (arrays assessing the in vivo effects of IFN-β are referred to as ex vivo experiments). The in vitro gene expression profile also served as a tool to identify genes of interest that might not have been regulated strongly enough ex vivo to meet the threshold criteria of significant regulation. The study presented herein was designed to identify the ex vivo gene expression pattern in six treatment responders as assessed by MRI (Stone et al., Neurology 49:862-9, 1997), a sensitive and well-established marker for multiple sclerosis disease activity, along with clinical disease activity. Two patients who initially responded to IFN-β but lost their response after developing high titers of neutralizing antibodies (NAbNR) and two patients who failed to respond fully to therapy from the beginning (initial non-responders, INRs) were also included.

### Example 1

### Patients and Methods

A. Patients and Patient Samples

All multiple sclerosis patients included in the current study participated in a longitudinal study with IFN-β-1b (Betaseron, Berlex, N.J., USA) as described previously (Stone et al., Ann Neurol 37:611-9, 1995; Wandinger et al., 2001, supra) and were followed up by the National Institute of Neurological Disorders and Stroke/Neuroimmunology Branch (NINDS)/NIB outpatient clinic. They had clinically-definite RR multiple sclerosis and were monitored by monthly clinical examinations and brain MRI (1,5-T MR unit; General Electric, Milwaukee, Wis., USA). New and total gadolinium (Gd)-DTPA-enhancing lesions (Magnevist; Berlex Laboratories, Cedar Knolls, N.J., USA) were assessed. The study was reviewed and approved by the NINDS Institutional Review Board, and all patients gave written informed consent. Ten female multiple sclerosis patients were identified as fulfilling the predetermined criteria for selecting their samples for cDNA array experiments. The criteria included:

(i) clinically definite multiple sclerosis; (ii) monitoring before and after start of IFN therapy by monthly MRI and clinical evaluations; (iii) no immunomodulatory therapy other than IFN for 30 days prior to obtaining blood sample; (iv) no MRI activity above average baseline activity at the baseline sampling time point; (v) no acute deterioration or relapse 30 days prior to sampling time point; (vi) no concurrent infection; (vii) 6 months or more on therapy (the NAbNR samples before developing NAb were collected at earlier time points); and (viii) sufficient peripheral blood mononuclear cell (PBMC) sample available during baseline and treatment to perform cDNA array experiments.

Patients were categorized based on clinical and MRI findings as: (i) treatment responders with clinically stable multiple sclerosis and a >60% reduction in mean number of total Gd-enhancing lesions compared with baseline (six patients); (ii) loss of MRI response related to development of high titers of neutralizing antibodies (NAb) to IFN-β (NAbNR, two patients); or (iii) failure to respond optimally from initiation of therapy as assessed by clinical and MRI measures (INR, two patients).

The indication to treat with IFN was based on MRI activity mainly in patients 1, 3, 4, 6 and NAbNR 2, and on both relapse and MRI activity in patients 2, 5, NAbNR1 and INR1 and 2.

PBMCs from these patients were collected as described previously (Wandinger et al., 2001, supra) at regular intervals before and during treatment with IFN-β, and stored in liquid nitrogen. Samples were taken within 48 hours of an IFN-β injection. For treatment responders, at least two time points were compared for the cDNA microarray experiment, i.e. baseline and treatment. For patients developing NAb, three time points were compared: baseline, treatment before NAb (or at low NAb) and treatment at time points of high NAb titers. For three of the responders (1, 2 and 3) and for the two NAb patients (treatment time point before NAb) two arrays were performed for different treatment time points that were evaluated as pairs to identify changes in gene regulation. Altogether, 10 baseline cDNA arrays were performed, eight arrays testing the in vitro response to IFN-β, nine for testing the ex vivo profile in responders, and eight arrays testing the ex vivo response in NAbNR and INR. It should be noted that this can be performed with any array, including high density arrays.

B. Microarrays and PCR

Cells stored in cryo-medium (20% dimethyl sulphoxide, 20% human serum albumin, HSA, 60% T cell medium) were thawed and viability determined by Trypan Blue exclusion (to be in the range of >80%). After in vitro incubation and for the ex vivo experiments the cells were spun down immediately after thawing, washed in T cell medium again, and transferred to RNeasy lysis buffer (RNeasy Kit; Qiagen, Santa Clarita, Calif., USA) for further processing. For the in vitro cDNA microarray testing, 108 PBMCs from baseline time points were incubated for 24 hours in 25 ml of T cell medium (Wandinger et al., 2001, supra). IFN-β (100 IU/ml) was added for testing of the in vitro effects at baseline. For the ex vivo testing of in vivo effects, 2×106 and 108 PBMCs were processed for PCR and microarray experiments, respectively. Total RNA was prepared by the RNA Midikit™ method (Qiagen) with yields of 40-90 μg. The same amount of RNA was used on arrays to compare conditions within a patient and a reference RNA pool (prepared from stimulated and unstimulated PBMC) was used on each array to allow comparisons of relative up- and downregulation of genes across experiments. The in vitro untreated baseline experiment was used as the comparator for both the in vitro incubation with IFN-β and the in vivo effect.

Mini-Lymphochip (MLC) cDNA microarrays were prepared from PCR-amplified material (Staudt and Brown, 2000, supra). cDNA probes were prepared, and microarray analysis of gene expression was essentially performed as described in Staudt and Brown (2000) and Alizadeh et al. (2000), using MLC versions that contained either 6432 or 12 672 array elements. Microarrays were analyzed on a GenePix scanner (Axon Instruments, Inverurie, Scotland), and data files were entered into a custom database maintained at the National Institutes of Health (see the NCI array database, available on the internet). Data was extracted for clustering analysis (programs ‘Cluster’ and ‘TreeView’ by M. Eisen, Stanford, Calif., USA) according to the following requirements: spot size of at least 25 μm, minimum intensities of 100 relative fluorescent units (RFU) in the Cy3 and Cy5 channels or minimum intensity of at least 1000 RFU in one of the two channels; spots labeled as ‘not found’ or ‘bad’ by the software were excluded. Relative levels of gene expression were calculated as the ratio of normalized intensities of the Cy5 and the Cy3 signal. Gene expression was ‘normalized’ by calculating the difference between expression at baseline and expression after in vitro incubation or in vivo treatment and displayed as a factor for up- and downregulation and as a color-coded matrix of genes. A two-fold increase or decrease compared with the baseline condition was accepted as clear indication of up- or downregulation of a gene. Only annotated and sequence-confirmed genes were searched and analyzed by the following algorithms.

Ex vivo and in vitro regulation of a given gene was considered relevant if at least three ex vivo (responders) or in vitro (responders and NAb patients before developing NAb) cDNA arrays had a two-fold change in expression, respectively, and if none of the responders showed a significant regulation in the opposite direction (separately assessed for in vitro and ex vivo findings). The latter rule was applied to control for the number of chance findings; it might, however, lead to the exclusion of some true variability within the expression profiles of IFN-β responders. If more than one out of the four arrays for non-responder status (INR and NAbNR) or one out of less than four arrays showed a significant regulation in the same direction as the responders, the gene was considered regulated also in non-responder status.

Real-time RT-PCR was employed according to a previously described method (Wandinger et al., 2001, supra) to confirm the findings for IL-8, a key cytokine detected through cDNA arrays as being markedly down-regulated ex vivo and in vitro by IFN treatment. cDNA was taken from the same sample that had been used for the cDNA array (for three responders, one INR and the two NAbNR), and additional time points at baseline and under treatment as well as additional patients were tested to allow for follow-up over several treatment time points for which not enough material was available to perform array experiments.

C. Statistics

To explore the ex vivo expression pattern a score sum counting genes regulated by a factor of 2 as ‘1’ and the absence of regulation as ‘0’ was used, and two-sided t-test statistics' were applied to compare responder and non-responder status. Imputation was performed for missing values using a ‘worst case’ approach in replacing missing expression values by ‘1’ for non-responders and by ‘0’ for responders. To analyze the RT-PCR findings for IL-8, mean values per patient over time were used and a two-sided t-test was performed.

### Example 2

### MRI Findings and Relapse Activity

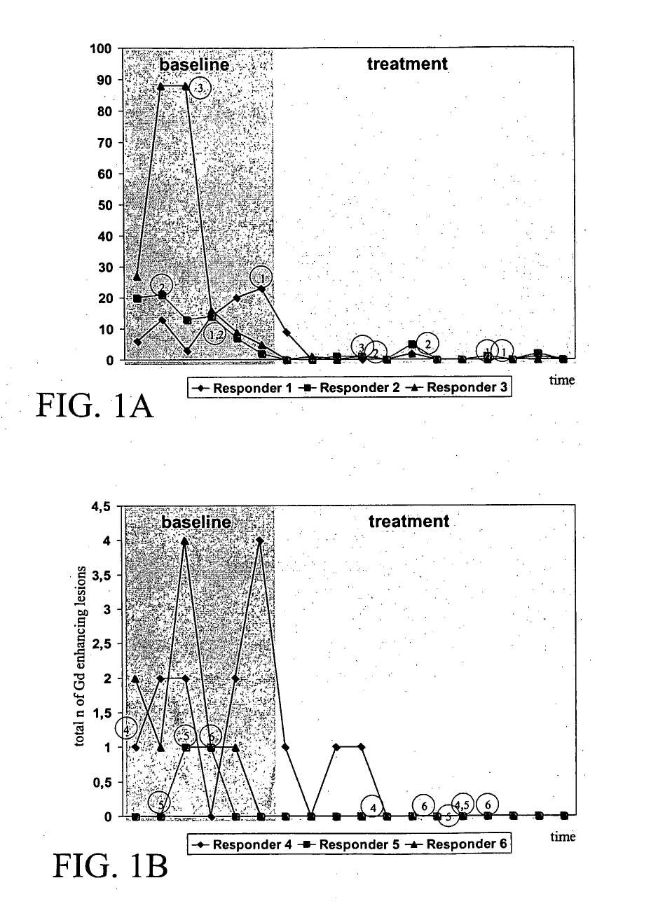

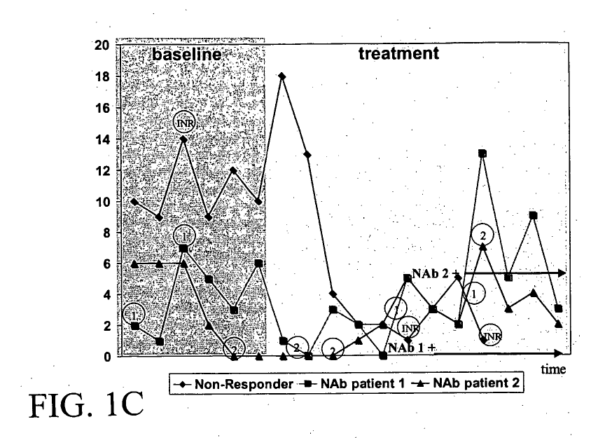

The responder status of the patients was defined by a reduction of at least 60% in the number of total Gd-enhancing lesions versus baseline (Stone et al., Neurology 49:862-9, 1997) and the marked reduction or absence of clinical disease activity (FIG. 1A-D). Accordingly, responders showed a marked reduction in the total number of Gd-enhancing lesions. Patients who developed NAb had a reduction in MRI activity preceding the development of NAb followed by reoccurrence of disease activity after developing NAb (titers of 549 and 477, respectively) (FIG. 1D). With the two INRs, both failed to demonstrate a (optimal) response with regard to the MRI criteria (FIG. 1C) from the onset of therapy. In the second INR, frequent relapses occurred in addition to fluctuating MRI activity (five relapses in the first 12 months of treatment). Two subgroups could be identified among the cohort of responders with regard to baseline MRI activity, i.e. patients with high numbers of Gd-enhancing lesions (FIG. 1A) and patients with markedly lower activity (FIG. 1B). More profound changes in gene expression appeared in those patients with high disease activity at baseline although no formal stratification was attempted due to the small number of individuals.

### Example 3

### cDNA Array Experiments

In vitro and ex vivo cDNA array data were obtained from eight of 10 patients (five of six responders, two of two NAb patients, and one of two INRs); in two patients, one responder and one INR, only ex vivo treatment samples could be examined due to the lack of sufficient cell material to perform the in vitro IFN incubation experiment. There were 112 genes identified that had a significant regulation by IFN-β; 25 genes were found significantly regulated ex vivo and an additional 87 genes in vitro. One likely explanation for the differences between the findings in ex vivo and in vitro experiments is the dose-dependency of gene expression changes; the concentration chosen for the in vitro incubation dose was 5-fold that of peak plasma levels after IFN-β administration in vivo. It must be taken into account that the 24 hour in vitro incubation and the ex vivo expression profile will not reflect all regulated genes, since a number of genes regulated early on after IFN-β have been added/administered will no longer show a transcriptional response.

Twenty of 25 (80%) of the ex vivo-regulated genes also showed a significant effect upon in vitro incubation with IFN-β. Eighty-eight percent of the genes regulated ex vivo in responders were not regulated during the non-responder status of the NAbNR patients or in the INRs. For 14 of 25 genes none of the non-responders showed a regulation, and for another eight genes only one out of four non-responders showed regulation.

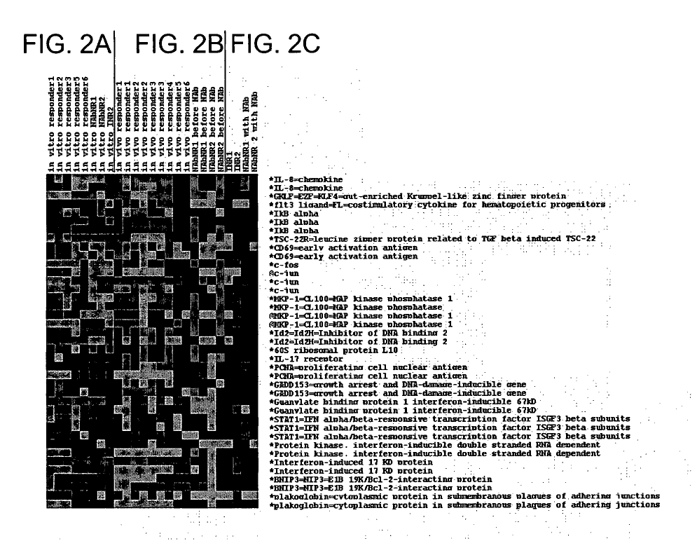

t-tests were performed to evaluate statistically the regulation pattern of all 25 genes together, and downregulated and upregulated genes separately. P values of 0.025 for all genes and for upregulated genes support a statistical significant difference between responders and non-responders; a P value of 0.083 for downregulated genes alone is indicative of a trend. Given the semi-quantitative nature of array findings, the results are presented as direction of regulation in Tables 1 and 2. Genes regulated ex vivo are depicted in FIG. 2.

With regard to the NAbNR patients there are two findings of particular importance: only four genes that were significantly regulated in responders ex vivo were found in both NAbNR patients before developing antibodies, and 10 of 25 genes were not regulated in any of the NAbNR during this time period. On the other hand, the in vitro pattern of regulation does not differ from the findings in responders. Given the small number of patients and the finding that not all genes are regulated significantly in the six responders either (see Table 1), these ex vivo findings should not be misinterpreted as being predictive of later NAb development.

TABLE 1
IFN-β-regulated genes ex vivo

| | | |
|---|---|---|
| | Responder | Non-responder* |
| | (no./arrays | (no./arrays |
| Genes regulated ex vivo | assessable) | assessable) |

Downregulation

| | | |
|---|---|---|
| AREB6 | 3/6 | 1/4 |
| CD69 | 3/5 | 0/4 |
| c-fos | 3/6 | 0/4 |
| c-jun | 3/6 | 0/4 |
| flt3 ligand | 3/6 | 0/3 |
| GADD 153 | 3/6 | 0/4 |
| GKLF | 4/6 | 0/4 |
| Id2 inhibitor of DNA binding | 3/6 | 0/4 |
| IκB-alpha | 4/6 | 1/4 |
| IL-8 | 4/6 | 1/4 |
| IL-17 receptor | 3/6 | 0/4 |
| IEX-1L | 4/6 | 1/4 |
| MKP1 | 4/6 | 0/4 |
| PCNA⁺ | 3/6 | 0/4 |
| 60S ribosomal protein | 3/6 | 0/4 |
| TSC-22R | 3/5 | 0/3 |

Upregulation

| BNIP3 | 3/5 | 0/4 |
|---|---|---|
| Dihydrofolate reductase | 3/4 | 1/3 |
| Guanylate-binding protein 1 | 4/6 | 1/4 |
| IFN-inducible 67 kDa | | |
| IFN-induced 17 kDa protein | 5/6 | 1/4 |
| 2′,5′ OAS | 5/6 | 2/4 |
| Plakoglobin | 3/6 | 1/4 |
| Proteinkinase, IFN inducible | 3/6 | 0/4 |
| STAT-1 | 3/6 | 1/4 |
| TRAIL | 3/4 | 2/2 |
| AREB6 = zinc finger homeodomain protein; flt3 ligand = costimulatory cytokine for hematopoietic progenitors; GADD 153 = growth arrest and DNA damage inducible gene; IκB-alpha = NF-κB inhibitory protein; IEX-1L = immediate early gene (apoptosis inhibitor); MKP1 = MAP kinase phosphatase 1; PCNA⁺ = proliferating cell nuclear antigen positive; | | |
| # TSC-22R (transforming growth factor beta stimulated clone 22 related gene) (protects T-cells from activation-induced apoptosis); BNIP3 = Bcl-2-interacting | | |
| # protein = pro(apoptotic mitochondrial protein). | | |
| *If more than one out of four, or one out of less than four arrays for non-responder status (NAb and INR patients) shows a significant regulation in the same direction as the responders, the gene is considered regulated in non-responder status. Genes that are regulated in the respective number of responders by a factor of ≧4 (log 2 = factor 2) are shown in bold. Numbers indicate up- or | | |
| # downregulation out of the number of assessable arrays for the respective gene. ‘+’ indicates regulation in vitro in the opposite direction to findings ex vivo. | | |

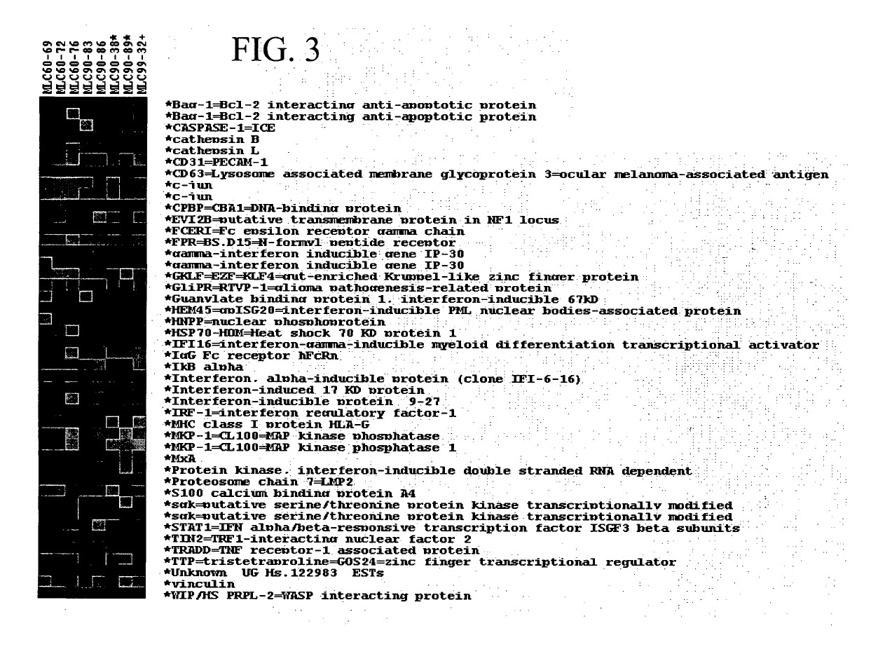

Out of the 87 genes that were significantly regulated in vitro, 59% were found in five or more out of the seven patients tested, thus showing a highly consistent expression profile. It was therefore decided to include the in vitro data from the NAbNR patients since their failure to respond was secondary to the development of NAb rather than a primary non-responsiveness. The in vitro response at baseline of eventually NAb-positive patients and the one INR for whom in vitro data are available do not substantially differ from the responder patients (FIG. 3).

### Example 4

### Detailed Analysis of Gene Expression Changes Upon IFN-β Treatment Ex Vivo Findings

The cytokines IL-8 and flt-3 ligand (fms-like tyrosine kinase 3 gene) (co-stimulatory cytokine for hematopoietic progenitors) were found significantly down-regulated ex vivo and in vitro. For these two genes no response was found for the INR and in one of the NAbNR patients. The marked reduction of IL-8 in responders ex vivo by a factor of ⅓ to 1/20 and the lack of an inhibitory effect in non-responders is a novel observation in multiple sclerosis. IL-8 is one of the important chemotactic mediators recruiting neutrophils to sites of inflammation, and IFN-β has been shown to inhibit IL-8 expression in vitro via an nuclear factor (NF)-B binding site (Oliveira et al., Mol Cell Biol 14:5300-8, 1994). IL-8 has been described not to differ in its expression in unstimulated PBMC from multiple sclerosis patients and normal volunteers (Jalonen et al., J Neurol 249: 76-83, 2002; Comabella et al., J Neuroimmunol 126:205-12, 2002). However, the increased expression of IL-8 in CD14+ cells after stimulation has been described to be inhibited by IFN-β ex vivo and in vitro in multiple sclerosis patients (Comabella et al., J Neuroimmunol 126:205-12, 2002).

A number of genes that are involved in the regulation of proliferation were downregulated by IFN treatment ex vivo and in vitro. The most prominent effect ex vivo was found for GKLF4 (gut-enriched Kruppel-like zinc finger protein). c-fos, c-jun and flt-3 (see above under cytokines) were downregulated, indicating an anti-proliferative effect of treatment. The downregulation of GKLF AREB6 (transcription factor, zinc finger domain protein), Id2 inhibitor of DNA binding, MKP-1 (MAP kinase phosphatase 1), and of IB-alpha may have opposing effects. One gene, PCNA (proliferating cell nuclear antigen), was downregulated ex vivo but showed an opposite effect after in vitro incubation. For this group of genes the lack of regulation in the NAbNR and the INR is evident with only one of the genes showing a change in expression in one of four non-responders.

Five apoptosis-related genes were regulated ex vivo and four of five [BNIP 3, TRAIL, IEX-1L (immediate early gene, apoptosis inhibitor), TSC22-R (transforming growth factor beta stimulated clone 22 related gene)] favor a pro-apoptotic state. In the INR and the NAbNR, only one of five genes showed regulation ex vivo. CD69, as an unspecific marker of T cell activation, was downregulated ex vivo and in vitro.

The ex vivo upregulation of expression of five IFN marker genes (2′5′ OAS, guanylate binding protein 1, STAT1, IFN-induced 17 kDa protein, IFN-induced protein kinase) in responders serves as a positive control for the expected biological effects. Only one of these genes was found regulated in non-responders.

### Example 5

### In Vitro Findings

Table 2 summarizes data for gene products that were only regulated in vitro.

TABLE 2
In vitro regulation of genes with no significant response pattern
ex vivo (including in vitro baseline findings for NAb patients)

| Functional class of | | |
|---|---|---|
| genes | Upregulation | Downregulation |
| Chemokines, cytokines, | CCR-1, GM-CSF/IL-5/IL-3 | HM74, N-formyl- |
| and their receptors and | receptor common β chain, | peptide-receptor |
| intracellular cascade | IP-10, IFN-γ inducible | |
| | IP-30, MCP-1, MIF, | |
| | MIP-1β, thioredoxin | |
| Macrophage activation | | Allograft |
| and inactivation | | inflammation |
| | | factor-1, IgG Fc |
| | | receptor |
| Co-stimulatory molecules | CD40 | |
| IFN-induced marker genes | AIM2, HEM45, IFN-induced | |
| | guanylate-binding protein | |
| | 2, IFN-inducible protein | |
| | 1-8, IFN-α-inducible | |
| | protein (clone IFI6-16), | |
| | IFN-inducible protein 9- | |
| | 27, IRF-1, IRF-7, MxA, | |
| | MxB, similar to IFN-γ- | |
| | inducible MG11, STAT- | |
| | induced STAT inhibitor-1 | |
| Antigen presentation and | MHC class I = HLA-A2, | |
| processing | MHC class I protein HLA-G, | |
| | myosin-IC, proteosome | |
| | chain 7 | |
| Control of cell cycle | DUSP5, MEF2A, retinoic | BTG2, FGR |
| and proliferation, | acid-induced RIG-E | tyrosine kinase, |
| kinases and phosphatases | precursor, sgk, VRK2 | JNK1, Jun B, |
| | kinase | Nak1, PAC-1, PKC |
| | | beta, p19, |
| | | ribosomal protein |
| | | L27 |
| Regulation of T-cell | Cathepsin B, CD13, CD38, | Vinculin |
| activation | CD59, CD63, CD72 | |
| Regulation of | IκB-epsilon, LYSP-100, | TTP (mRNA |
| transcription | NF-κ B-p65, STAT6, | stability |
| | TNF-α-inducible TRAF1, | regulating |
| | 2-binding inhibitor of | factor) |
| | NF-κB activation | |
| Adhesion molecules | CD54 | CD11c, CD18, |
| | | CD31, CD50 |
| Matrixmetalloproteinases | TIMP-1 | |
| Apoptosis -related genes | Bag-1, Caspase-1, | GADD45 beta |
| | Caspase-3, CD-95, c-IAP2, | |
| | TRAF signaling complex | |
| | protein, FLAME 1, TRADD | |
| Others | Acid finger protein, | EVI2B, GLiPR, |
| | aspartyl tRNA synthetase | Oncostatin M, |
| | alpha 2 subunit, | similar to |
| | Cathepsin L, hSEC10p, | microsomal |
| | homolog of mouse MAT-1 | dipeptidase |
| | oncogene, MDS01, hSTYXb, | precursor = |
| | metallothionein II, NAD- | dehydropeptidase, |
| | dependent | ribosomal protein |
| | methylenetetrahydrofolate | L5, ribosomal |
| | dehydrogenase, similar to | protein S5, |
| | 52 KD RO protein | ribosomal protein |
| | | S9 |
| AIM2 = interferon inducible protein AIM2 (absent in melanoma); HEMAS = interferon stimulated gene 20 kDa (degrades single stranded RNA); DUSP5 = dual specificity phosphatase induced by serum; MEF2A = MADS/MEF2 (myocyte enhancer-binding factor); sgk = putative serine/threonine protein kinase; VRK2 kinase = vaccina virus kinase related kinase; Bag-1 = Bcl-2 interacting anti-apoptotic protein; c-IAP2 = inhibitor of apoptosis; | | |
| # FLAME 1 = FLICE-like inhibitory protein long form (anti-apoptotic molecule); TRADD = TNF receptor-1-associated protein; hSEC10p = brain secretory protein; MDS01 = phorbolin I paralogue; hSTYXb = tyrosine phosphatase-like protein; HM74 = G protein coupled receptor; | | |
| # BTG2 = p53 dependent anti-proliferative gene; JNK1 = stress-activated protein kinase; Nak1 = nuclear receptor subfamily 4, group A (orphan steroid receptor); PAC-1 = protein tyrosine phosphatase; p19 = cyclin-dependent kinase 4 inhibitor; TTP = tristetraproline; GADD 45 beta = growth arrest and DNA damage inducible protein; EVI2B = ecotropic viral integration site 2 B protein (putative transmembrane protein in NF 1 locus); | | |
| # GLiPR = glioma pathogenesis-related protein. | | |

The upregulation of pro-inflammatory chemokines MIP-1β, IP-10, MCP-1 and six other genes related to chemokine and cytokine pathways indicates a pro-inflammatory shift of monocyte- and lymphocyte-derived mediators, respectively. The most prominent changes are found for IP-10 with 5.6- to 174-fold and MCP-1 with 12.6- to 160-fold increases in expression. The in vitro regulation of 13 further gene products that are involved in cell-cycle control supports the notion of an overall anti-proliferative effect of IFN-β. The findings for IP-10 and MCP-1 should be related to the findings of Comabella et al. (2002), supra, who found an inhibitory effect of IFN-β on ex vivo and in vitro stimulated PBMCs. The differences may be explained by the additional stimulation used by Comabella et al. (anti-CD3 plus anti-CD28). The in vitro findings indicate a shift toward a pro-apoptotic state of gene expression.

Gene products involved in antigen presentation (four genes) were only found to be regulated (upregulation) in vitro. Two markers of macrophage activation were downregulated. The influence on adhesion molecule expression (CD11c, CD18, CD31, CD50 and CD54) showed one gene product being upregulated and four downregulated. The pattern of regulation for cathepsin B, CD13, CD38, CD63, CD59 and CD72 is interpreted as representing a ‘balanced’ change with regard to either stimulation or inhibition of immune responses, although such conclusions are difficult to draw from array data only. The co-stimulatory molecule CD40 was upregulated, favoring a pro-inflammatory state. The in vitro upregulation of 12 genes known as IFN marker genes (see Table 2; IFN-induced marker genes) serves as a positive control of the expected biological response to IFN.

Previous findings (Wandinger et al., 2001, supra) regarding CCR-5 and IL-12 receptor β2 chain were only partly confirmed here, (i) because the gene for IL-12 receptor β2 chain was not represented on the array used and (ii) only two of the in vitro experiments showed a significant signal for CCR-5, while ex vivo arrays showed no measurable signal.

### Example 6

### Confirmation by PCR

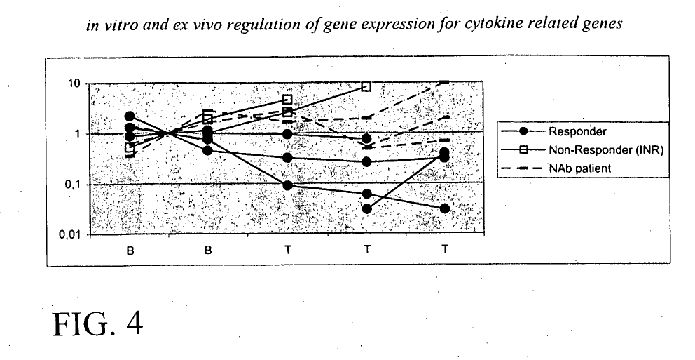

The marked changes in IL-8 expression were followed by quantitative real-time RT-PCR studies in four responders, two INR and three NAbNR (including three responders, the INRs and the two NAbNRs from the array series, see FIG. 4) for time points under treatment from 2 to 22 months. Whereas all responders showed a consistent suppression of gene expression for IL-8 over time, an increased expression was found in the INRs and no effect in the NAbNRs once high titers of NAb occurred. Exploratory statistics based on the mean percentage change during treatment versus baseline as the measure of effect, showed a significant difference between responders and the group of INR and NAbNR (P=0.035).

Current understanding of the pathogenesis of multiple sclerosis indicates that it represents a heterogeneous group of disorders based on clinical, MRI, pathological, immunological and genetic findings (Lucchinetti et al., Ann Neurol 47:707-17, 2000; Stürzebecher et al., Neuroimaging Clin N Am 10:649-68, 2000). Available treatments such as IFN-β exert a multitude of effects on various tissues and cell types, and it can be expected that not all of these are beneficial for the disease process in multiple sclerosis if considered as single markers. The lack of a uniform treatment response in the entire population of multiple sclerosis patients, even if only one stage, i.e. RR multiple sclerosis, is considered suggests that not all patients respond similarly at the molecular level to the dose levels of IFN-β achievable in vivo, and that the net outcome of these effects will determine the clinical results and the extent to which inflammation in the CNS is inhibited. With respect to the mechanism of action of IFN-β, the data demonstrate that it induces a number of anti-inflammatory, but also pro-inflammatory, activities (Wandinger et al., 2001, supra), and it is likely that it is the balance of many moderate or even small changes in different biological pathways that leads to clinical benefit.

On the other hand, a single cytokine can have dual or multiple effects, such as depending on the level of expression, the activation status of the immune system or the site of action. This could apply to these findings of an increase of pro-inflammatory cytokines (in vitro) that may, if relevant in vivo, exhaust the cellular response in the periphery and thereby have a protective effect on the inflammatory response in the CNS. Tumor necrosis factor (TNF)- is another important example (Sharief and Hentges, New Engl J Med 325:467-72, 1991). A wealth of evidence indicated that TNF- is involved in the pathogenetic process of multiple sclerosis and that its inhibition would benefit patients. Contrary to these expectations and different from the experiences in the treatment of rheumatoid arthritis, blocking TNF- has, however, worsened disease in multiple sclerosis patients (Lenercept Multiple Sclerosis Study Group and The University of British Columbia MS/MRI Analysis Group, Neurology 53:457-65, 1999).

The rapid progress in the area of genomics, particularly the study of the expression of thousands of genes by cDNA or oligonucleotide microarrays, has enabled the examination of these questions as described herein. Using this technology, responses of very complex systems can be analyzed to assess the response of cells and tissues in their interactions. In the work described herein, gene expression profiling was used to capture not only the complex immunomodulatory activity of IFN-β in individual multiple sclerosis patients, but furthermore to relate the biological response of a number of informative genes to the therapeutic response to IFN-β treatment. These experiments show that individual responder and non-responder profiles or sets of marker genes can be identified in IFN-β-treated multiple sclerosis patients. Based on a well accepted disease activity marker that is useful for visualizing the response to IFN-β (Stone et al., Ann Neurol 37:611-9, 1995, Stone et al., Neurology 49:862-9, 1997), the total number of Gd-enhancing MRI lesions, and the clinical disease activity, patients were classified as: (i) IFN-β responders (>60% reduction of MRI disease activity); (ii) patients who initially responded, but later developed NAb and then lost IFN-β responsiveness; and (iii) two patients who never fully responded to IFN-β (INR). The ex vivo response of patients of group (ii) and (iii) was meant to provide a ‘negative control;’ there was a clear trend for ex vivo gene regulation that distinguishes this group of non-responding patients from the responders with most of the genes of interest being not regulated. The in vitro response to treatment in patients later developing NAbs documents that these patients are capable of responding to IFN-β and do not differ at baseline from the responders. The same appears to be correct in INRs.

Thus, the gene expression profile in response to IFN-β in vitro and in vivo is complex and includes influences on cell migration, matrix degradation, proliferation, cell cycle control, cell differentiation, antigen processing and presentation, apoptosis, and cytokine and chemokine regulation. Secondly, even though the ex vivo response pattern reflects only part of the in vitro effects of IFN-β on gene expression, 25 out of the 112 genes that are modified are regulated by at least about factor of two ex vivo. The evaluation of the ex vivo response assessed after a short treatment interval can be used to predict long-term treatment response in the context of, for example, frequent MRI examination early during treatment. The data with IL-8 represent an example of how single genes potentially related to treatment response can be identified using the cDNA array approach. The results on IL-8 appear rather clear with regard to distinguishing responders from non-responders. However, the responder state can be identified not just by individual genes, but by the expression of groups of genes. Arrays capturing the expression of large numbers of genes, enabling the examination of all relevant biological responses to a treatment, can be used to develop individualized therapies, such as to determine an appropriate dose of IFN-β-1b, the appropriateness of the use of different drugs, or the likelihood of success of combination therapies.

### Example 7

### Exemplary Nucleic Acid Sequence

Expression of a variety of nucleic acid sequences can be assessed using the methods disclosed herein. Exemplary nucleic acid sequences are provided in Table 3.

TABLE 3
IFN-β-regulated genes ex vivo, Accession Number, SEQ ID NO:

| | | |
|---|---|---|
| | Accession | SEQ ID |
| Genes regulated ex vivo | Number | NO: |

Downregulation

| | | |
|---|---|---|
| AREB6 | D15050 | 1 |
| CD69 | Z22576 | 2 |
| c-fos | K00650 | 3 |
| c-jun | J04111 | 4 |
| flt3 ligand | U03858 | 5 |
| GADD 153 | S40706 | 6 |
| GKLF | AF105036 | 7 |
| Id2 inhibitor of DNA binding | NM_002166 | 8 |
| IκB-alpha | AF080157 | 9 |
| IL-8 | M68932 | 10 |
| IL-17 receptor | NM_014339 | 11 |
| IEX-1L | AF071596 | 12 |
| MKP1 | NM_002745 | 13 |
| | NM_138957 | 14 |
| PCNA⁺ | AF527838 | 15 |
| 60S ribosomal protein | ? | ? |
| TSC-22R | AF153603 | 16 |

Upregulation

| BNIP3 | U15174 | 17 |
|---|---|---|
| Dihydrofolate reductase | NM_000791 | 18 |
| Guanylate-binding protein 1 IFN-inducible 67 | NM_002053 | 19 |
| kDa | | |
| IFN-induced 17 kDa protein | X84958 | 20 |
| 2′,5′ OAS | NM_003733 | 21 |
| | NM_198213 | 22 |
| Plakoglobin | NM_002230 | 23 |
| | NM_021991 | 24 |
| Proteinkinase, IFN inducible | AH006648 | 25 |
| STAT-1 | NM_007315 | 26 |
| TRAIL | NM_139266 | 27 |
| | NM_003810 | 28 |
| AREB6 = zinc finger homeodomain protein; flt3 ligand = costimulatory cytokine for hematopoietic progenitors; GADD 153 = growth arrest and DNA damage inducible gene; IκB-alpha = NF-κB inhibitory protein; IEX-1L = immediate early gene (apoptosis inhibitor); MKP1 = MAP kinase phosphatase 1; PCNA⁺= proliferating cell nuclear antigen positive; TSC-22R (transforming growth factor beta stimulated | | |
| # clone 22 related gene) (protects T-cells from activation-induced apoptosis); BNIP3 = Bcl-2-interacting protein = pro(apoptotic mitochondrial protein). | | |
| *If more than one out of four, or one out of less than four arrays for non-responder status (NAb and INR patients) shows a significant regulation in the same direction as the responders, the gene is considered regulated in non-responder status. Genes that are regulated in the respective number of responders by a factor of ≧4 (log 2 = factor 2) are shown in bold. Numbers indicate up- or downregulation out of the number of assessable | | |
| # arrays for the respective gene. ‘+’ indicates regulation in vitro in the opposite direction to findings ex vivo. | | |

Other therapeutic protocols can be evaluated using the methods disclosed herein. These include any therapy that aims at disease modification that targets specific and pathogenetically relevant process.

It will be apparent that the precise details of the methods or compositions described may be varied or modified without departing from the spirit of the described invention. We claim all such modifications and variations that fall within the scope and spirit of the claims below.

1. A method for determining if a subject with multiple sclerosis will respond to a therapeutic protocol, comprising:
creating a cDNA probe from mRNA of lymphocytes isolated from the subject
hybridizing the probe to a microarray comprising gene sequences
determining the extent of hybridization of the probes to each gene on the microarray
wherein a pattern of hybridization of the probes on the microarray indicates that the subject with multiple sclerosis will respond to the therapeutic protocol.

2. The method of claim 1, wherein the therapeutic protocol comprises treatment with interferon beta.

3. The method of claim 1, wherein the therapeutic protocol comprises treatment with an antibody that specifically binds the interleukin-2 receptor.

4. The method of claim 2, wherein determining the extent of hybridization of the probes comprises evaluating the expression of IL-8.

5. The method of claim 1, wherein determining the extent of hybridization of the probes comprises the evaluation of the expression of Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL.

6. The method of claim 1, wherein determining the extent of hybridization of the probes comprises the evaluation of the expression of zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R).

7. A method for determining if a subject with multiple sclerosis will respond to a therapeutic protocol, comprising:
hybridizing cDNA probes created from mRNAs of cells isolated from the subject to a microarray comprising nucleic acid sequences, wherein the microarray comprises the nucleic acid sequences encoding IL-8, Bcl-2-interacting protein (BNIP3), dihydrofolate reductase, gyanylate-binding protein 1, interferon-induced 17 kDa protein, 2′5′ OAS, plakoglobin, interferon inducible protein kinase, STAT-1, TRAIL, zinc finger homeodomain protein, CD 69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R);
determining an extent of hybridization of the probes to nucleic acid sequences on the microarray;
wherein a pattern of hybridization of the probes to the microarray indicates that the subject with multiple sclerosis will respond to the therapeutic protocol.

8. The method of claim 7, wherein determining the extent of hybridization of the probes to each gene on the microarray comprises detecting a decrease in expression of a mRNA from a lymphocyte from the subject as compared to the expression of the mRNA a lymphocyte from subject not treated with the therapeutic protocol.

9. The method of claim 7, wherein determining the extent of hybridization of the probes to each gene on the microarray comprises detecting an increase in expression of a mRNA from a cell from the subject as compared to the expression of the mRNA a cell from subject not treated with the therapeutic protocol.

10. The method of claim 7, wherein the therapeutic protocol comprises treatment with interferon beta.

11. The method of claim 7, wherein the therapeutic protocol comprises treatment with an antibody that binds the interleukin-2 receptor.

12. A method of determining if a subject with multiple sclerosis will respond to treatment with interferon-beta, comprising
contacting a lymphocyte from the subject with interferon-beta in vivo or in vitro;
detecting a decrease in expression of interleukin-8 of a lymphocyte from the subject as compared to the expression of interleukin-8 by a lymphocyte from subject not treated with interferon-beta;
wherein a decrease in interleukin-8 expression demonstrates that the subject will respond to treatment with interferon beta.

13. The method of claim 12, further comprising detecting an increase of expression of at least one of Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL.

14. The method of claim 12, further comprising detecting an increase of expression of at least two of Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL.

15. The method of claim 12, further comprising detecting an increase of expression of detecting an increase of expression of at least one of Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, TRAIL.

16. The method of claim 13, further comprising detecting a decrease of expression of at least one of zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R).

17. The method of claim 13, further comprising detecting a decrease of expression of at least two of zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R).

18. The method of claim 13, further comprising detecting a decrease of expression of zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, and transforming growth factor beta stimulated clone 22 related gene (TSC-22R).

19. A method of determining if a subject with multiple sclerosis will respond to treatment with interferon-beta, comprising
treating a cell from the subject with interferon-beta in vivo or in vitro;
detecting an increase in expression of at least one protein or at least one mRNA encoding the protein from a cell isolated from the subject as compared to the expression of the protein or the mRNA by a cell isolated from subject not treated with interferon-beta, wherein the at least one protein comprises Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL;
wherein an increase in expression of the protein or mRNA following a treatment with interferon-beta demonstrates that the subject will respond to treatment with interferon beta.

20. The method of claim 19, further comprising detecting an increase in expression of an additional protein or an additional mRNA encoding the protein from a cell isolated from the subject as compared to the expression of the protein or the mRNA by a cell isolated from subject not treated with interferon-beta, wherein the additional protein comprises Bcl-2-interacting protein (BNIP3), dihydrofolate reductase, gyanylate-binding protein 1, interferon-induced 17 kDa protein, 2′5′ OAS, plakoglobin, interferon inducible proteinkinase, STAT-1, or TRAIL,
wherein the additional protein is not identical to the at least one protein, and wherein increase in expression of the at least one protein or mRNA and the additional protein or mRNA following a treatment with interferon-beta demonstrates that the subject will respond to treatment with interferon beta.

21. The method of claim 19, further comprising detecting a decrease of expression of at least one other protein or mRNA encoding the other protein, wherein the other protein comprises zinc finger homeodomain protein, CD69, c-fos, costimulatory cytokine for hematopoietic progenitors (flt-3 ligand), growth arrest and DNA damage inducible gene, GKLF, Id2 inhibitor of DNA binding, NF-κB inhibitory protein, IL-8, IL-17 receptor, immediate early gene (apoptosis inhibitor), MAP kinase phosphatase 1, proliferating cell nuclear antigen positive, 60S ribosomal protein, or transforming growth factor beta stimulated clone 22 related gene (TSC-22R),
wherein decreased expression of the other protein or mRNA encoding the other protein demonstrates that the subject will respond to treatment with interferon beta.
